(19) United States
(12) Patent Application Publication
(10) Pub. No.: US 2022/0106357 A1
(43) Pub. Date: Apr. 7, 2022

(54) PLASMA FRACTIONATION UTILIZING SPRAY-DRIED HUMAN PLASMA

(71) Applicant: Takeda Pharmaceutical Company Limited, Osaka (JP)

(72) Inventors: Zhorzh Patatanyan, Glendale, CA (US); Rohit Murthy, West Hollywood, CA (US); Yasser Baddour, Rolling Hills Estates, CA (US); Alexander Zaydenberg, Chatsworth, CA (US)

(21) Appl. No.: 17/492,256

(22) Filed: Oct. 1, 2021

Related U.S. Application Data
(60) Provisional application No. 63/086,335, filed on Oct. 1, 2020.

(51) Int. Cl. C07K 1/14; C07K 14/745; C07K 16/00; C07K 14/765; C07K 14/81; C07K 14/755

(52) U.S. Cl. CPC C07K 1/145; C07K 14/745; C07K 14/755; C07K 14/765; C07K 14/8125; C07K 16/00

(57) ABSTRACT

The present invention provides a method of fractionating human plasma, in some embodiments, using the Cohn fractionation procedure. The improvement comprises the use of physiologically active reconstituted spray dried human plasma as the starting material for the fractionation procedure.

### CROSS REFERENCE TO RELATED APPLICATIONS

This application claims priority to U.S. Provisional Patent Application No. 63/086,335, filed Oct. 1, 2020, entitled “PLASMA FRACTIONATION UTILIZING SPRAY-DRIED HUMAN PLASMA,” which is hereby incorporated by reference in its entirety.

### FIELD OF THE INVENTION

The present invention resides in the field of plasma fractionation to separate therapeutically active proteins from plasma.

### BACKGROUND OF THE INVENTION

To facilitate storage and transportation of blood plasma until fractionation, plasma is typically preserved by freezing soon after its collection from a donor. Fresh-Frozen Plasma (FFP) is obtained through a series of steps involving centrifugation of whole blood to separate plasma and then freezing the collected plasma within less than 8 hours of collecting the whole blood. Alternatively, plasma is collected from donors using plasmapheresis equipment, in which the blood cells are separated from plasma and returned to the donor. In the United States, the American Association of Blood Banks (AABB) standard for storing FFP is up to 12 months from collection when stored at a temperature of −18° C. or below. FFP may also be stored for up to 7 years from collection if maintained at a temperature of −65° C. or below. European standards dictate that FFP has a shelf life of 3 months if stored at temperatures between −18° C. to −25° C., and for up to 36 months if stored below −25° C. Under European standards thawed plasma must be transfused immediately or stored at 1° C. to 6° C. and transfused within 24 hours. If stored longer than 24 hours, the plasma must be relabeled for other uses or discarded.

Thus, FFP must be maintained in a temperature-controlled environment throughout its duration of storage to prevent degradation of certain plasma proteins, adding to the difficulty and cost and difficulty of storage and transport. Furthermore, FFP must be thawed prior to use, resulting in a delay of 30-80 minutes before it may be used after removal from cold storage. Clearly, a method dispensing with the need for a cold-storage chain for plasma pre-fractionation would represent a significant advance in the fractionation of the 23 to 28 million liters of plasma fractioned each year. Burnouf, Transfus. Med. Rev. (2007); 21(2): 101-117.

A possible solution for eliminating the need for maintaining plasma in a frozen state has relied on lyophilized plasma. Dried blood products are known in the art, and the predominant technique for achieving the dried product is lyophilization (freeze-drying). For example, U.S. Pat. Nos. 4,287,087 and 4,145,185 to Brinkhous et al. disclose dried blood platelets that have been fixed with a crosslinking reagent such as formaldehyde. U.S. Pat. Nos. 5,656,498; 5,651,966; 5,891,393; 5,902,608; and 5,993,804 disclose additional dried blood products. Such products are useful for therapeutic purposes because they are stable, have long shelf life, and can be used potentially in powder form to arrest bleeding in patients undergoing severe trauma. However, fractionation of reconstituted lyophilized plasma is not suggested in these references.

Introducing spray dried plasma into the fractionation process has the potential to eliminate the need for the pre-fractionation cold-chain. Spray-drying is a technology in which a solution is atomized in a stream of flowing gas for rapid solvent vaporization (e.g., dehydration). The result is the formation on a sub-second timescale of microparticles composed of the residual solute. Spray-drying has been used as an industrial process in the material, food, and pharmaceutical industries for decades. More recently, spray-drying has facilitated the preparation of protein therapeutics as microparticles for inhalation (Maltesen, et al., Eur J Pharm Biopharm 70, 828-838 (2008)).

Reconstitutable, spray dried whole plasma has been used in trauma settings and on the battlefield. Though less than ideal, it finds utility in its storablity in a wide range of environments without freezers or refrigerators, its availabity for use by first responders at the initial point of care, and it can be transfused in minutes without the 30-45 minute delay associated with thawing of frozen plasma.

Though a potentially attractive expedient, the spray drying process, under certain conditions and parameters, can harm the plasma proteins. Spray drying subjects plasma proteins to high stress forces during the aerosolization process as the plasma is forced through a narrow orifice exposed to high rate of air flow that is necessary to create suitably sized droplets for drying. Second, the spray drying process exposes plasma proteins to high temperatures necessary to force the water from the aerosolized droplets. Third, the spray drying process subjects the plasma proteins to dramatic and rapid increases in pH as a result of the rapid release of CO₂ during drying.

The spray drying process, depending on the parameters, can reduce amounts of certain large multimeric proteins (e.g., von Willebrand factor (vWF)), degrade large proteins into smaller protein fragments, and/or affect the activity/functionality of proteins. As the goal of plasma fractionation is the isolation (or enrichment) of physiologically functional plasma proteins into various fractions, one of ordinary skill in the art would not look to nor find suggestion or motivation in the spray drying or lyophilization art with regard to incorporating spray dried plasma as the starting material for plasma fractionation to prepare intact, physiologically active protein pharmacological agents.

Accordingly, until the invention described herein, it has not been apparent that the proteins in the various fractions (e.g., cold ethanol fractions) could be recovered by fractionating reconstituted spray dried plasma in amounts sufficiently meaningful to make the expense of fractionating the reconstituted physiologically active plasma worthwhile. Additionally, it was not known whether the reconstituted physiologically active spray dried plasma would act similarly to fresh frozen plasma in Cohn Fractionation (or a known modification thereof). The inventors have discovered that this fractionation route is indeed feasible and have devised an economically viable Cohn Fractionation or Kistler-Nitschman Fractionation, or other method (e.g., Gerlough, Hink, and Mulford methods) commencing with reconstituted spray dried plasma. See, e.g., Kistler et al., Vox. Sang. (1962); 7(4), pp. 414-424; Graham, et al. Subcellular Fractionation, a Practical Approach. Oxford University Press. 1997.

### BRIEF SUMMARY OF THE INVENTION

Given the broad use of therapeutic plasma-derived blood protein compositions, such as immune globulin compositions, albumin, protease inhibitors, blood coagulation factors, coagulation factor inhibitors, and proteins of the complement system, ensuring adequate, economical, environmentally friendly, and sustainable access to efficacious and safe plasma-derived blood protein compositions is of paramount importance.

In 2019, the blood plasma product market was forecast to grow at a CAGR of 6.8% to reach $28.5 B in 2023 from $20.5 B in 2018. The global annual fractionation capacity was about 70.7 million liters in 2016. Frozen plasma is transported from donor centers to fractionation centers. Cold chain spending in biopharma, of which the plasma fractionation industry is a sector, was estimated in 2020 to be about $17.2 B, up from 2019's $15.7 B. “2020 Biopharma Cold Chain Sourcebook forecasts a $17.2-billion logistics market”—Pharmaceutical Commerce, Apr. 27, 2020. Clearly, the economic and environmental impact of storing and transporting many millions of liters of frozen plasma, maintained under refrigeration, continues to be a significant consideration in the plasma industry. See, e.g., Robert P, Hotchko M. Worldwide 2016 Plasma Protein Sales—Marketing Research Bureau, Inc. Published Dec. 1, 2017.

The present invention ameliorates these and other problems by providing a plasma fractionation process originating with physiologically active spray dried plasma. In addition to providing efficacious and safe compositions, the present invention provides a process for isolating vital plasma proteins using a plasma source that accesses components of the cold chain less intensively, and is simpler and more economical to transport from donor centers to fractionation facilities than liquid plasma.

With the current invention, it has quite surprisingly been discovered that physiologically active spray dried, and reconstituted plasma is an efficacious starting material for preparing protein therapeutic agents by fractionating the physiologically active reconstituted plasma. In various embodiments, the proteins typically found in the various Cohn fractions downstream from the physiologically active spray dried plasma are found in these fractions in yields and purity comparable to those found in corresponding fractions in a process starting with frozen plasma.

An exemplary method of the invention includes: providing a physiologically active reconstituted plasma solution prepared by reconstituting physiologically active spray dried plasma powder in a reconstitution liquid; and submitting the physiologically active reconstituted plasma to one or more plasma fractionation processes (e.g., cold ethanol fractionation).

The physiologically active spray dried plasma has the advantages of a long storage life at room temperature or standard refrigeration; easy storage and shipment due to its reduced weight and volume; versatility, durability and simplicity, and it can be easily and rapidly reconstituted and used at the site of fractionation. The physiologically active spray dried plasma preferably can be stored at least about 2-3 years at virtually any temperature (e.g., −180° C. to 40° C.). U.S. Publication 2019/0298765. The costs associated with storage and shipping of the physiologically active spray dried plasma are significantly lower than those for liquid plasma, because of its lighter weight and broader range of temperature tolerance compared to frozen plasma.

The physiologically active spray dried plasma of use in the present invention can be produced in either a batch (single unit) or a continuous (e.g., pooled units) process mode.

The present invention also provides a plasma processing system, preferably a cGMP compliant system, which is used, inter alia, to fractionate plasma introduced into the fractionation process by means of a reconstituted spray dried, physiologically active plasma powder solution. The starting physiologically active spray dried plasma can be dried from plasma directly into a final, attached sterile container, which can later be transferred to a reconstitution tank where the dried plasma it is rapidly and easily reconstituted into state and concentration appropriate for fractionation. At the fractionation site, the physiologically active spray dried plasma can be rapidly reconstituted

### BRIEF DESCRIPTION OF THE DRAWINGS

FIG. 1 is a generalized flow diagram of an exemplary Cohn fractionation procedure.

FIG. 2. Is a diagram of an exemplary spray drying device of use in practicing the current invention.

FIG. 3 is a table displaying the coagulation factor activity for thawed plasma derived from FFP for several coagulation factors. Physiologically active spray dried plasma powder of the type described herein may exhibit substantially similar coagulation activity for one or more or all of the listed factors. (2019/0298765).

FIG. 3 provides exemplary steps in a model spray drying run, and data derived from reconstitution and analysis of a composition of the invention.

FIG. 4 is a tabulation of parameters for exemplary spray dry runs on plasma samples.

FIG. 5A and FIG. 5B, together are a tabulation of results from a post-reconstitution analysis such as that described in Example 2.

FIG. 6 is exemplary flow diagrams for two different fractionation processes starting with spray dried plasma starting material, TEST 1 and TEST 2, detailed in Example 3 and FIG. 7A-7D.

FIG. 7A-7D is a tabulation of results from TEST 1, TEST 2 and TEST 3 (initiated at Fraction V).

### DETAILED DESCRIPTION OF THE INVENTION

### I. Introduction

Making up about 55% of the total volume of whole blood, blood plasma is a whole blood component in which blood cells and other constituents of whole blood are suspended. Blood plasma further contains a mixture of over 700 proteins and additional substances that perform functions necessary for bodily health, including clotting, protein storage, and electrolytic balance, amongst others. When extracted from whole blood, blood plasma may be employed to replace bodily fluids, antibodies and clotting factors. Accordingly, blood plasma is extensively used in medical treatments.

Currently millions of liters of plasma are fractionated per year in a process requiring a cold chain for the plasma from the collection center to the fractionating site with the frozen plasma being stored in freezers, and thawed immediately before fractionation. Maintenance of the cold-chain during shipping of the plasma from the collection sites to the fractionation site is a logistically complex, resource intensive, expensive element of the plasma fractionation process and business that could be improved by innovations focusing on sustainability. Elimination of the cold-chain or a component of the cold-chain results in an increase in technological and economic efficiency, and a “greener”, more sustainable process.

As set forth in the following sections, the present invention, by starting fractionation with reconstituted physiologically active spray dried plasma, imparts numerous efficiencies and other advantages to the fractionation process.

Reference will now be made in detail to implementation of exemplary embodiments of the present disclosure as illustrated in the accompanying drawings. The same reference indicators will be used throughout the drawings and the following detailed description to refer to the same or like parts. Those of ordinary skill in the art will understand that the following detailed description is illustrative only and is not intended to be in any way limiting. Other embodiments of the present disclosure will readily suggest themselves to such skilled persons having benefit of this disclosure.

In the interest of clarity, not all of the routine features of the implementations described herein are shown and described. It will be appreciated that, in the development of any such actual implementation, numerous implementation-specific decisions are made in order to achieve the plasma product producer's specific goals, such as compliance with application- and business-related constraints, and that these specific goals will vary from one implementation to another and from one plasma product producer to another. Moreover, it will be appreciated that such a development effort might be complex and time-consuming, but would nevertheless be a routine undertaking of engineering for those of ordinary skill in the art having the benefit of this disclosure.

Many modifications and variations of the exemplary embodiments set forth in this disclosure can be made without departing from the spirit and scope of the exemplary embodiments, as will be apparent to those skilled in the art. The specific exemplary embodiments described herein are offered by way of example only, and the disclosure is to be limited only by the terms of the appended claims, along with the full scope of equivalents to which such claims are entitled.

### II. Abbreviations and Definitions

Unless defined otherwise, all technical and scientific terms used herein generally have the same meaning as commonly understood by one of ordinary skill in the art to which this invention belongs. Generally, the nomenclature used herein and the laboratory procedures in organic chemistry, pharmaceutical formulation, and medical imaging are those well-known and commonly employed in the art.

### a. Abbreviations

“aPTT”, as used herein refers to Activated Partial Thromboplastin Time, a performance indicator known in the art measuring the efficacy of both the “intrinsic” (sometimes referred to as the contact activation pathway) and the common coagulation pathways.

“PT”, as used herein, refers to Prothrombin Time, a performance indicator known in the art of the extrinsic pathway of coagulation.

“FGN”, as used herein, refers to Fibrinogen (also referred to in the art as Factor I), an insoluble plasma glycoprotein, synthesized by the liver, that is converted by thrombin into fibrin during coagulation.

“PC”, as used herein, refers to Protein C, also known as autoprothrombin HA and blood coagulation Factor XIV.

“PS”, as used herein, refers to Protein S, a vitamin K-dependent plasma glycoprotein synthesized in the endothelium. In the circulation, Protein S exists in two forms: a free form and a complex form bound to complement protein C4b. In humans, protein S is encoded by the PROS1 gene.

As used herein, a “Factor” followed by a Roman Numeral refers to a series of plasma proteins which are related through a complex cascade of enzyme-catalyzed reactions involving the sequential cleavage of large protein molecules to produce peptides, each of which converts an inactive zymogen precursor into an active enzyme leading to the formation of a fibrin clot. They include: Factor I (fibrinogen), Factor II (prothrombin), Factor III (tissue thromboplastin), Factor IV (calcium), Factor V (proaccelerin), Factor VI (no longer considered active in hemostasis), Factor VII (proconvertin), Factor VIII (antihemophilic factor), Factor IX (plasma thromboplastin component; Christmas factor), Factor X (Stuart factor), Factor XI (plasma thromboplastin antecedent), Factor XII (hageman factor), and Factor XIII (fibrin stabilizing factor).

“FP24” refers to frozen plasma prepared from a whole blood collection and must be separated and placed at −18° C. or below within 24 hours from whole blood collection. The anticoagulant solution used and the component volume are indicated on the label. On average, units contain 200 to 250 mL. This plasma component is a source of non-labile plasma proteins. Levels of Factor VIII are significantly reduced and levels of Factor V and other labile plasma proteins are variable compared with FFP. This plasma component serves as a source of plasma proteins for patients who are deficient in or have defective plasma proteins. Coagulation factor levels might be lower than those of FFP, especially labile coagulation Factors V and VIII.

### b. Definitions

The articles “a” and “an” are used herein to refer to one or to more than one (i.e., to at least one) of the grammatical object of the article. By way of example, “a protein” means one protein or more than one protein.

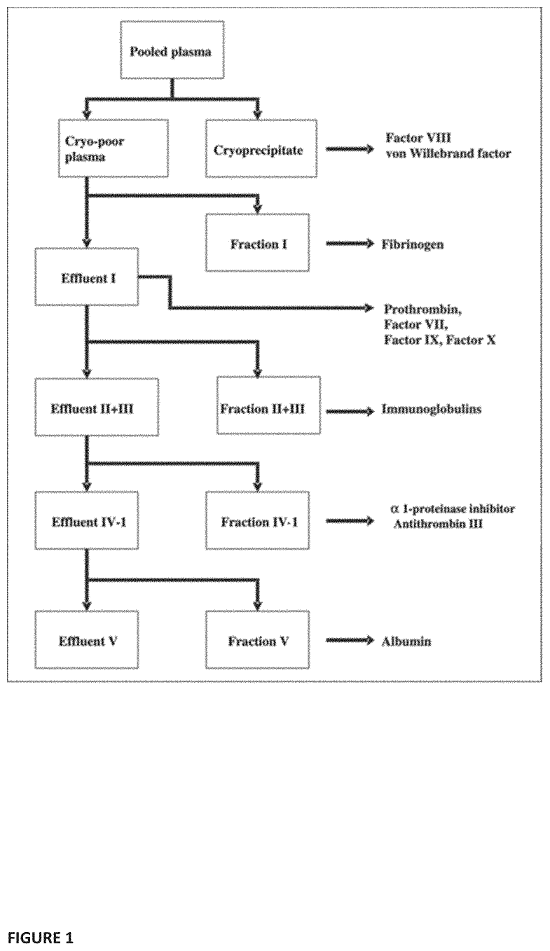

The “Cohn Process”, and “Cohn Fractionation” are used interchangeably herein and as generally understood, refer to a method of separating human plasma through a series of steps, including ethanol precipitation at differing concentrations, changes in pH, changes in temperature, changes in ionic strength, which lead to fractions enriched in certain plasma proteins. See, for example U.S. Pat. No. 2,390,074. FIG. 1 provides an exemplary flow diagram for the Cohn Process. As used herein, the terms “Cohn Process” and “Cohn Fractionation” also refers to the many variations and improvements on this pioneering process, e.g., Kistler-Nitschmann Process (Kistler et al. (1952), Vox Sang, 7, 414-424). Other processes of use in the methods of the invention include the method of isolating IgG set forth in U.S. Pat. No. 8,940,877

“Plasma” is the fluid that remains after blood has been centrifuged (for example) to remove cellular materials such as red blood cells, white blood cells and platelets. Plasma is generally yellow-colored and clear to opaque. Blood that is donated and processed to separate the plasma from the other certain blood components, and not frozen is referred to as “never-frozen” plasma. Plasma that is frozen within 8 hours to temperatures, described herein, is referred to herein as “fresh frozen plasma” (“FFP”). It contains the dissolved constituents of the blood such as proteins (6-8%; e.g., serum albumins, globulins, fibrinogen, etc.), glucose, dotting factors (clotting proteins), electrolytes (Na⁺, Ca²⁺, Mg²⁺, HCO₃⁻, Cl⁻, etc.), hormones, etc. Whole blood (WB) plasma is plasma isolated from whole blood with no added agents except anticoagulant(s). Citrate phosphate dextrose (CPD) plasma, as the name indicates, contains citrate, sodium phosphate and a sugar, usually dextrose, which are added as anticoagulants.

“Liquid plasma” refers to plasma other than spray dried plasma.

“Recovered plasma” refers to plasma separated no later than 5 days after the expiration date of the Whole Blood and is stored at 1 to 6° C. The profile of plasma proteins in Liquid Plasma is poorly characterized. Levels and activation state of coagulation proteins in Liquid Plasma are dependent upon and change with time in contact with cells, as well as the conditions and duration of storage. This component serves as a source of plasma proteins. Levels and activation state of coagulation proteins are variable and change over time.

“Thawed plasma” refers to plasma derived from FFP or FP24, prepared using aseptic techniques (closed system), thawed at 30 to 37° C., and maintained at 1 to 6° C. for up to 4 days after the initial 24-hour post-thaw period has elapsed. Thawed plasma contains stable coagulation factors such as Factor II and fibrinogen in concentrations similar to those of FFP, but variably reduced amounts of other factors.

“Fresh frozen plasma” (“FFP”) refers to plasma prepared from a whole blood or apheresis collection and frozen at −18° C. or colder within the time frame as specified in the directions for use for the relevant blood collection, processing, and storage system (e.g., frozen within eight hours of draw). On average, units contain 200 to 250 mL, but apheresis derived units may contain as much as 400 to 600 mL. FFP contains plasma proteins including all coagulation factors. FFP contains high levels of the labile coagulation Factors V and VIII.

As used herein, the term “spray dried plasma” refers to physiologically active plasma powder which, when reconstituted, includes proteins that have not been damaged to such an extent to lose substantially all of their physiological activity. The physiological activity of a plasma powder, in its reconstituted form, may by indicated by a number of parameters known in the art including, but not limited to: Prothrombin Time (PT), Activated Partial Thromboplastin Time (aPTT), Fibrinogen level, Protein C level, and Protein S level. The physiological activity of a plasma powder, in its reconstituted form, may be indicated by coagulation factor levels or other protein activities known in the art including, but not limited to: Factor II, Factor V, Factor VII, Factor VIII, Factor IX, and Factor X; fibrinogen activity; IgG antigen binding activity; A1PI activity; antithrombin III activity; alpha-2-antiplasmin activity; and alpha-1-anti-trypsin activity. These parameters may be measured using techniques known in the art, e.g., using commercially available instruments. An exemplary spray dried plasma is dried by the methods described in U.S. Pat. Nos. 8,601,712; 8,595,950; 8,533,972; 8,533,971; 8,434,242; and 8,407,912.

As used herein, the term “physiologically active reconstituted plasma”, and variations of this term refer to a reconstituted physiologically active spray dried plasma powder, which include proteins that have not been damaged by spray drying and/or reconstitution to such an extent to lose substantially all of their physiological efficacy in a therapeutic regimen in which the protein(s) is/are administered to treat a disease in a subject in need of such treatment. In an exemplary embodiment, the physiologically active reconstituted spray dried plasma retains at least about 30%, at least about 40%, or at least about 50% of the clotting factor activity of the plasma before spray drying and reconstitution. In some embodiments, the physiologically active reconstituted spray dried plasma retains from about 30%, to about 70%, from about 40% to about 60% of the clotting factor activity of the plasma before spray drying and reconstitution. In various embodiments, the IgG activity of the physiologically active reconstituted plasma is not less than 50%, not less than 60%, not less than 70%, not less than 80%, not less than 90%, not less than 95%, not less than 99% that of the IgG activity of the plasma before spray drying.

The physiological activity of one or more components of a spray dried plasma powder, in its reconstituted form, is determined by standard tests and indicated by a number of parameters known in the art including, but not limited to: Prothrombin Time (PT), Activated Partial Thromboplastin Time (aPTT), Fibrinogen level, Protein C level, and Protein S level. The physiological activity of a plasma powder, in its reconstituted form, may be indicated by coagulation factor levels or other protein activities known in the art including, but not limited to: Factor II, Factor V, Factor VII, Factor VIII, Factor IX, and Factor X; ; fibrinogen activity; IgG antigen binding activity; A1PI activity; antithrombin III activity; alpha-2-antiplasmin activity; and alpha-1-anti-trypsin activity.

A “reconstitution liquid” is an aqueous liquid with which the physiologically active spray dried plasma powder is contacted to bring the powder into solution/suspension, forming “reconstituted plasma” (i.e., physiologically active reconstituted plasma). A reconstitution solution can include one or more salt, one or more buffer, one or more amino acid, one or more suspending agent, and the like, and in any useful combination. Exemplary additives in the reconstitution liquid are selected for their ability to stabilize the proteins in the liquid and prevent, diminish or retard damage to the proteins and/or loss of protein activity during the reconstitution process. Exemplary reconstitution liquids include water for injection, sodium phosphate buffer, acetate buffer, aqueous solutions including one or more physiologically acceptable surfactant (e.g., Polysorbate 80), and those which are described in U.S. Publications 2017/0370952; 2017/0370952; and 2010/0273141.

A “disease” is a state of health of an animal wherein the animal cannot maintain homeostasis, and wherein if the disease is not ameliorated then the animal's health continues to deteriorate. In various embodiments, one or more proteins from the fractionated reconstituted physiologically active spray dried plasma are used to treat one or more disease.

### III. EMBODIMENTS

### A. Compositions and Devices

Embodiments of the present disclosure are directed to methods of fractionating physiologically active plasma reconstituted from spray dried plasma, and protein preparations prepared by this fractionation.

In an exemplary embodiment, the invention provides one or more plasma fraction, which is a product of a plasma fractionation process commencing with reconstituted physiologically active spray dried plasma. In an exemplary embodiment, the fraction is a Cohn fraction as this term is understood in the art. In another embodiment, the invention provides a solution of physiologically active plasma reconstituted from spray dried plasma using a reconstitution liquid selected to allow, facilitate or promote subsequent fractionation of the reconstituted plasma. In various embodiments, a physiologically active reconstituted plasma solution is disposed in a reconstitution tank that is in line with one or more additional component used in plasma fractionation. In an exemplary embodiment, the reconstituted physiologically active plasma in the reconstitution tank is a component of a fractionation system. In an exemplary embodiment, the fractionation system is a Cohn fractionation system, or a known modification of this system.

In various embodiments, the invention provides one, two, three, four, five or more unique plasma fraction composition(s) downstream from a physiologically active reconstituted dried plasma starting material. In an exemplary embodiment, the composition is cryopaste and/or cryo poor plasma. In various embodiments, the composition is Fraction I paste and comprises fibrinogen, or Fraction I supernatant. In various embodiments, the composition is Fraction II +III paste and comprises IgG, or Fraction II+III supernatant. In some embodiments, the composition is Fraction IV-1 paste and comprises A1PI and/or AT-III, or Fraction IV-1 supernatant. In an exemplary embodiment, the composition is Fraction IV-4 paste and/or Fraction IV-4 supernatant. In various embodiments, the composition is Fraction V paste and comprises albumin, or Fraction V supernatant. In various embodiments, the fraction of the invention contains primarily FVIII and/or von Willebrand Factor. In some embodiments, the fraction of the invention includes primarily prothrombin and/or Factor VII, and and/or FIX and/or FX. In some embodiments, the fraction of the invention contains primarily IgG. In an exemplary embodiment, the fraction of the invention includes primarily A1PI and/or AT-III. In some embodiments, the fraction of the invention includes primarily albumin. In an exemplary embodiment, the fraction or fractions is/are one or more Cohn fraction.

In an exemplary embodiment, the invention provides a preparation of a coagulation factor produced by a method of the invention. In various embodiments, the preparation of the coagulation factor is selected from Factor VIII, Factor IX, prothrombin complex, von Willebrand factor, fibrinogen and a combination of any two or more thereof.

In some embodiments, the invention provides a preparation of polyvalent and/or hyperimmune immunoglobulins (IgGs) prepared by a method of the invention. In various embodiments, the IgG is selected from anti-RhO hyperimmune immunoglobulin, anti-hepatitis B hyperimmune immunoglobulin, anti-rabies hyperimmune immunoglobulin, anti-tetanus IgG hyperimmune immunoglobulin and a combination of any two or more thereof.

In an exemplary embodiment, the invention provides a preparation of a protease inhibitors prepared by a method of the invention. In various embodiments, the protease inhibitor is selected from alpha 1-antitrypsin, C1-inhibitor, etc.) and a combination thereof.

In an exemplary embodiment, the invention provides a preparation of one or more anticoagulant prepared by a method of the invention. In various embodiments, the preparation comprises antithrombin, e.g., AT-III.

In an exemplary embodiment, the invention provides a preparation of albumin prepared by a method of the invention.

In an exemplary embodiment, the fraction isolated according to the invention has characteristics substantially identical to those of the same fractions isolated in the same manner from frozen plasma using art-recognized methods. In various embodiments, the characteristics of the fraction vary from those of the same fractions isolated in the same manner from frozen plasma using art-recognized methods. In a preferred embodiment, the characteristics varying correspond to one or more parameter of regulatory relevance and the characteristic varies within a range of such one or more parameter by an amount considered insignificant to relevant regulatory requirements for that fraction, i.e., a pharmaceutical formulation incorporating a fraction or a protein isolated from a fraction does not require new regulatory consideration or marketing approval.

In an exemplary embodiment, the method provides an aqueous albumin solution containing at least 5% or at least 25% by volume of albumin and suitable for intravenous injection, which solution remains stable without precipitation of the albumin after exposure to a temperature of 45° C. for a period of one month. This solution is isolated by fractionation from a solution of physiologically active reconstituted spray dried human plasma.

In an exemplary embodiment, the invention provides a preparation of a protein in Cryopaste isolated from the physiologically active reconstituted spray dried human plasma selected from Factor VIII, Factor IX and a combination thereof. The preparation comprises the protein in an amount of not less than 80% of the yield in which this protein is isolated from fresh frozen plasma. In various embodiments, the activity of the protein is not less than 60%, 65%, 70%, 75%, 80%, 85%, 90%, or 95% of the activity of the protein isolated from fresh frozen plasma.

In an exemplary embodiment, the invention provides a preparation of IgG isolated from the physiologically active reconstituted spray dried human plasma. The preparation comprises the IgG in an amount of not less than 60%, 65%, 70%, 75%, 80%, 85%, 90%, or 95% of the amount found in an identical preparation in which IgG is isolated from fresh frozen plasma. In various embodiments, the activity of the IgG is not less than 60%, 65%, 70%, 75%, 80%, 85%, 90%, or 95% of the activity of the IgG isolated from fresh frozen plasma.

In an exemplary embodiment, the invention provides a protein isolated from Fraction IV-1 of the fractionated physiologically active reconstituted spray dried human plasma selected from A1PI, AT-III and a combination thereof is isolated in a yield of not less than 60%, 65%, 70%, 75%, 80%, 85%, 90%, or 95% of the yield in which this protein is isolated from fresh frozen plasma. In various embodiments, the protein isolated from the physiologically active reconstituted spray dried human plasma in Fraction IV-1 has an activity of not less than 60%, 65%, 70%, 75%, 80%, 85%, 90%, or 95% of the activity of the protein isolated from fresh frozen plasma.

In some embodiments, the invention provides a method wherein albumin isolated from Fraction V of the physiologically active reconstituted spray dried human plasma is isolated in a yield of not less than 80% of the yield in which this protein is isolated from fresh frozen plasma. In various embodiments, the albumin isolated from the physiologically active reconstituted spray dried human plasma has an activity of not less than 60%, 65%, 70%, 75%, 80%, 85%, 90%, or 95% of the activity of albumin isolated from fresh frozen plasma.

In various embodiments, the invention provides a pharmaceutical formulation comprising one of the fractions of the invention, or a protein component of one or more such fraction further purified from such fraction. Various pharmaceutical formulations also include a pharmaceutically acceptable vehicle in which the proteins in the fraction (or downstream where further purified) are formulated.

In various embodiments, the invention provides a pharmaceutical formulation of the invention packaged in a device for administering the pharmaceutical formulation to a subject in need of such administration, e.g., a syringe, infusion bag, and the like. In various embodiments, the device contains a unit dosage formulation of the active protein for administration to a subject in need of such administration. In an exemplary embodiment, the unit dosage is an art-recognized unit dosage for a subject.

### B. Methods

The present invention provides a novel method of plasma fractionation commencing with physiologically active reconstituted spray dried plasma as the starting material. An exemplary method of the invention includes: providing a physiologically active plasma solution prepared by reconstituting physiologically active plasma powder in a reconstitution liquid; and submitting the physiologically active plasma thus reconstituted to one or more fractionation process. An exemplary fractionation process is Cohn fractionation, Kistler Nitchman fractionation, and variations thereof. FIG. 1.

The spray dried plasma of use in the methods of the present invention may be dried after pooling or unit-by-unit. Pooling of multiple plasma units has some benefits. For example, any shortfall in factor recovery on an equal-volume basis can be made up by adding volume from the pool to the finished product. There are negative features as well. Making up volume from the pool to improve factor recovery is expensive. Importantly, pooled plasma must be constantly tested for pathogens as any pathogens entering the pool from, for example, a single donor, runs the risk of harming hundreds or thousands of patients if not detected.

In various embodiments, the spray dried plasma enters the plant for further processing, e.g., fractionation, in any convenient form. In an exemplary embodiment, the spray dried plasma enters the plant in a sealed container, e.g., a sealed plastic bag. The contents of the container are transferred to a reconstitution tank. In an exemplary embodiment, the transfer is performed in a clean room, or under other aseptic conditions. In some embodiments, the container is configured such that it can be attached to a port on the reconstitution tank and the spray dried plasma transferred directly to the reconstitution tank without exposure to the ambient plant atmosphere. In this configuration, the transfer can be performed in a clean room or outside this environment. The transfer can be facilitated by various powder transfer means, including mechanical (e.g., screws, vibrators), pneumatic and vacuum means.

In an exemplary embodiment, the plasma is contacted with one or more anticoagulant prior to spray drying. An exemplary anti-coagulant is a citrate salt, e.g., sodium citrate.

The physiologically active spray dried plasma powder is reconstituted in the reconstitution tank by contacting the powder with a reconstitution liquid. The contacting can be performed in any useful format (i.e., order of addition, temperature, dilution, agitation, etc.).

Proteins potentially undergo physical degradation by a number of mechanisms (e.g., clipping, oxidation, unfolding, aggregation, insoluble particulate formation). Many proteins are structurally unstable in solution and are susceptible to conformational changes due to various stresses encountered during purification, processing and storage. These stresses include temperature shift, exposure to pH changes and extreme pH, shear stress, surface adsorption/interface stress, and so on. An exemplary reconstitution liquid exerts a protective effect on one or more protein in the spray dried plasma, preventing or reducing degradation, aggregation, or other negative outcomes during reconstitution, thereby retaining physiological activity.

In one embodiment, at least a portion of the physiologically active spray dried plasma powder is added to the reconstitution tank, which previously was charged with at least a portion of the reconstitution liquid. In some embodiments, at least a portion of the reconstitution liquid is added to at least a portion of the physiologically active spray dried plasma powder, which has been loaded into the reconstitution tank. In either of these formats, the contents of the tank can be agitated by any convenient means at any point before, during or after contacting the powder and the reconstitution liquid. In an exemplary embodiment, the contents of the reconstitution tank are agitated by stirring.

One component of the reconstitution mixture (spray dried plasma or reconstitution liquid) is added to the other at a rate and in a volume that is determined to provide useful results in the reconstitution. Thus, one component can be added to the other residing in the reconstitution tank, slowly, quickly or in a bulk bolus.

In various embodiments, the plasma is reconstituted in the tank by contacting the stirred reconstitution liquid in the tank with the physiologically active spray dried plasma powder. The reconstitution liquid may be stirred or otherwise agitated. The physiologically active spray dried plasma powder can be added to the liquid quickly, slowly or in a bulk bolus.

In some embodiments, the reconstitution tank is charged with at least a portion of the physiologically active spray dried plasma powder to be reconstituted, and the powder is stirred or otherwise agitated. Alternatively, the physiologically active spray dried plasma physiologically active spray dried plasma powder is not stirred or otherwise agitated. The reconstitution liquid is added to the powder in the tank. Numerous modes of addition are of use, e.g., adding the liquid directly to the powder, or adding the liquid to the physiologically active spray dried plasma powder by pouring down the side walls of the tank. The liquid may be added quickly, slowly or in one or more bolus.

In various embodiments, at least a portion of the physiologically active spray dried plasma powder and at least a portion of the reconstitution liquid are added essentially simultaneously to the reconstitution tank, which may be empty or may already contain physiologically active spray dried plasma powder, reconstitution liquid or a combination thereof.

As will be appreciated by those of skill in the art, any of these modes of contacting can be performed singly or in any combination or order.

An exemplary reconstitution liquid is a physiologically compatible liquid.

The reconstitution fluid is an aqueous fluid that is capable of reconstituting the spray dried plasma and minimizing damage (e.g., denaturation, aggregation, loss of activity) to the protein components of plasma, and loss of or reduction in key plasma characteristics and activity(ies).

An exemplary reconstitution liquid is water for injection (WFI) or saline. In various embodiments, the pH of the reconstitution liquid is adjusted. As will be appreciated by those of skill in the art, the pH of the reconstitution liquid is readily adjusted by addition of acids and bases, e.g., HCl, sodium bicarbonate and the like. In various embodiments, the reconstitution liquid is one of these liquids and it is used without pH adjustment.

In some embodiments, the reconstitution liquid includes at least one buffer. Exemplary buffers are, without limitation, salts of phosphate, hydrogen phosphate, acetate, citrate, carbonate, bicarbonate and other such buffers generally recognized as being compatible with plasma proteins.

In various embodiments, the reconstitution liquid includes at least one amino acid. An exemplary amino acid is glycine.

In an exemplary embodiment, the reconstitution liquid includes one or more anticoagulant. An exemplary anti-coagulant is a citrate salt, e.g., sodium citrate.

A further advantage offered by the method of the invention is the ability to reduce the amount of liquid being processed by reconstituting the plasma at a higher protein concentration than is found in native plasma. In an exemplary embodiment, the spray dried plasma is reconstituted with the reconstitution liquid to about 100% of its original volume. In some embodiments, the spray dried plasma is reconstituted with the reconstitution liquid to about 75% of its original volume. In some embodiments, the spray dried plasma is reconstituted with the reconstitution liquid to about 50% of its original volume. In some embodiments, the spray dried plasma is reconstituted with the reconstitution liquid to about 25% of its original volume. In some embodiments, the spray dried plasma is reconstituted with the reconstitution liquid to from about 25% to about 50%, e.g., from about 30% to about 40% of its original volume. In some embodiments, the spray dried plasma is reconstituted with the reconstitution liquid to from about 50% to about 75%, e.g., from about 60% to about 70% of its original volume. In various embodiments, the spray dried plasma is reconstituted with the reconstitution liquid to about 20%, about 25%, about 30%, about 35%, about 40%, about 45%, about 50%, about 55%, about 60%, about 65%, about 70%, about 75%, about 80%, about 85%, about 90%, about 95% or about 100%.

In various embodiments, the physiologically active reconstituted plasma is composed of at least about 2%, 5%, 7%, 10%, 12%, 14%, 16%, 18%, or 20%. In some embodiments, when reconstituted at a ratio of 0.09 grams of powder to 1 mL of reconstituting fluid, the reconstituted physiological plasma has a protein concentration of about 48 mg/mL, e.g., in the range of 45-55 mg/mL.

Given the convenience provided by the method of invention of not requiring the use of frozen/thawed plasma, the reconstitution process can occur at any useful temperature. Exemplary reconstitutions occur at room temperature (e.g., from about 22° C. to about 25° C.), under refrigeration (from about 10° C. to about 20° C.). In an exemplary embodiment, the reconstitution process is performed at a temperature of from about 2° C. to about 28° C.

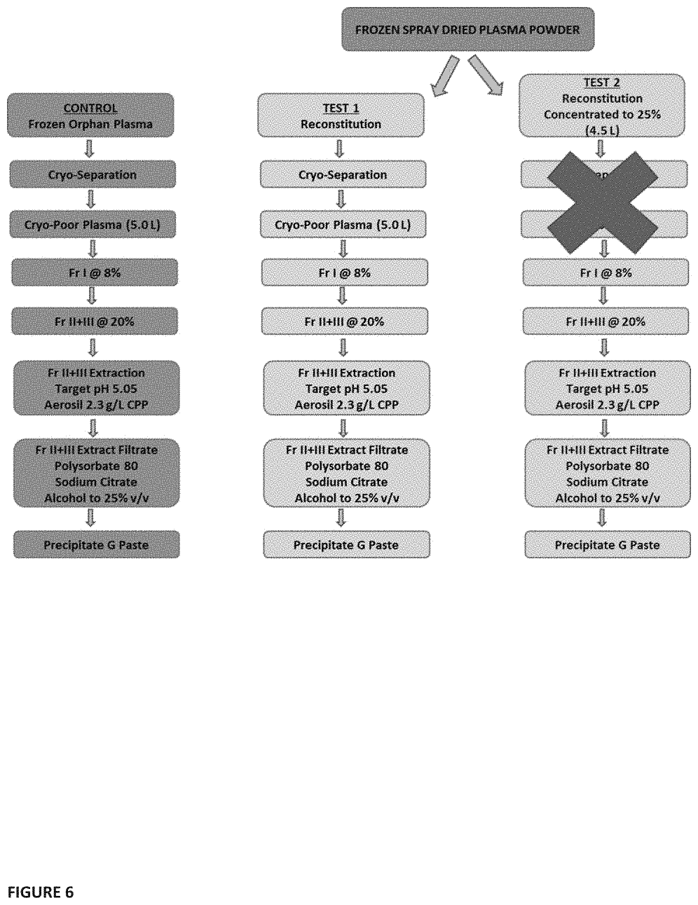

In an exemplary embodiment, following reconstitution, the temperature of the reconstituted plasma is lowered to promote cryoprecipitation and the cryoprecipitate and supernatant are separated. In various embodiments, the temperature of the reconstituted plasma is lowered to under about 6° C. to effect cryoprecipitation. In an exemplary embodiment, the reconstituted plasma solution is cooled to between from about 1° C. to about 6° C. FIG. 6.

In various embodiments, following cryoprecipitation the plasma is separated into cryoprecipitate and cryosupernatant. The cryosupernatant is optionally submitted to further fractionation steps. The separation may be accomplished in any useful fashion, such as, without limitation, centrifugation, filtration or a combination thereof.

In those embodiments in which cooling of the physiologically active reconstituted plasma is desired, any useful means of cooling can be utilized. In various embodiments, a vessel or line containing the reconstituted plasma is jacketed with a cooling device. In exemplary embodiments, the cooling and/or plasma solution is retained in a vessel, e.g., a jacketed vessel, and, in some embodiments, the plasma solution is cooled during inline flow (“radiator method”).

In various embodiments, cooling the physiologically active reconstituted plasma as discussed above results in fibrinogen precipitating. The precipitated fibrinogen can be separated from the supernatant. In some embodiments, fibronectin precipitates on cooling the reconstituted plasma and can be separated from the supernatant. In some embodiments, FVIII precipitates on cooling the physiologically active reconstituted plasma, and can be separated from the supernatant. In various embodiments, von Willebrand Factor precipitates on cooling the physiologically active reconstituted plasma and can be separated from the supernatant.

In an exemplary embodiment, the physiologically active reconstituted plasma is submitted to one or more testing procedure to confirm one or more activity prior to being fractionated. Activities of pro-coagulant and anti-coagulant proteins queried in the physiologically active reconstituted plasma, include but are not be limited to; the following tests: i. Prothrombin time (PT) or international normalized ratio (INR); ii. Activated partial thromboplastin time (aPTT); iii. Activity of heat-labile proteins (e.g., Factor V, Factor VIII); iv. Activity of anticoagulant proteins (e.g., Protein S, Protein C); v. Antigen and activity of large coagulation proteins prone to aggregation and degradation (e.g., fibrinogen, von Willebrand factor); vi. Markers of coagulation activation (e.g., thrombin-antithrombin complexes, fibrin degradation products)

In some embodiments, the physiologically active spray dried plasma powder, when reconstituted, exhibits physiological activity substantially equivalent to Thawed Plasma, Liquid Plasma, FP24, or FFP. In various embodiments, the plasma powder exhibits a recovery rate for plasma proteins between the starting, native plasma and the physiologically active reconstituted plasma, of at least 50%, at least 60%, at least 70%, at least 80%, at least 90%, etc. In some embodiments, the physiologically active reconstituted plasma has protein levels comparable to or better than FFP or FP24.

In some embodiments, the physiologically active spray dried plasma, when reconstituted, is characterized by an aPTT of about 65 seconds or less, a PT of about 31 seconds or less, and a Fibrinogen level of at least about 100 mg/dL.

In some embodiments, the physiologically active spray dried plasma, when reconstituted, is characterized by an aPTT of about 35 seconds or less, a PT of about 15 seconds or less, and a Fibrinogen level of at least about 223 mg/dL.

In some embodiments, the physiologically active spray dried plasma, when reconstituted, is characterized by an aPTT in the range of 28-66 seconds, a PT in the range of 14-31 seconds, and a Fibrinogen level in the range of 100-300 mg/dL.

In some embodiments, the physiologically active spray dried plasma, when reconstituted, is characterized by an aPTT in the range of 30-35 seconds, a PT in the range of 10-15 seconds, and a Fibrinogen level in the range of 223-500 mg/dL.

In some embodiments, the physiologically active spray dried plasma, when reconstituted, is characterized by at least one of: a Factor VII level of at least about 10 IU/dL, a Factor IX level of at least about 10 IU/dL, a Protein C level of at least about 10 IU/dL, and a Protein S level of at least about 10 IU/dL.

In some embodiments, the physiologically active spray dried plasma, when reconstituted, is characterized by at least one of: a Factor VII level of at least about 30 IU/dL, a Factor IX level of at least about 25 IU/dL, a Protein C level of at least about 55 IU/dL, and a Protein S level of at least about 54 IU/dL

In some embodiments, the physiologically active spray dried plasma, when reconstituted, is characterized by at least one of: a Factor VII level of at least about 54 IU/dL, a Factor IX level of at least about 70 IU/dL, a Protein C level of at least about 74 IU/dL, and a Protein S level of at least about 61 IU/dL.

In some embodiments, the physiologically active spray dried plasma, when reconstituted, is characterized by at least one of: a Factor VII level in the range of 30-110 IU/dL, a Factor IX level in the range of 25-135 IU/dL, a Protein C level in the range of 55-130 IU/dL, and a Protein S level of in the range of 55-110 IU/dL.

In some embodiments, the physiologically active spray dried plasma, when reconstituted, is characterized by at least one of: a Factor VII level in the range of 34-172 IU/dL, a Factor IX level in the range of 70-141 IU/dL, a Protein C level in the range of 74-154 IU/dL, and a Protein S level of in the range of 61-138 IU/dL.

In some embodiments, the physiologically active spray dried plasma, when reconstituted, is characterized by at least one of: a Factor V level of at least about 10 IU/dL, and a Factor VIII level of at least about 10 IU/dL.

In some embodiments, the physiologically active spray dried plasma, when reconstituted, is characterized by at least one of: a Factor V level of at least about 30 IU/dL, and a Factor VIII level of at least about 25 IU/dL.

In some embodiments, the physiologically active spray dried plasma, when reconstituted, is characterized by at least one of: a Factor V level of at least about 63 IU/dL, and a Factor VIII level of at least about 47 IU/dL.

In some embodiments, the physiologically active spray dried plasma, when reconstituted, is characterized by at least one of a Factor V level in the range of 63-135 IU/dL, a Factor VIII level in the range of 47-195 IU/dL.

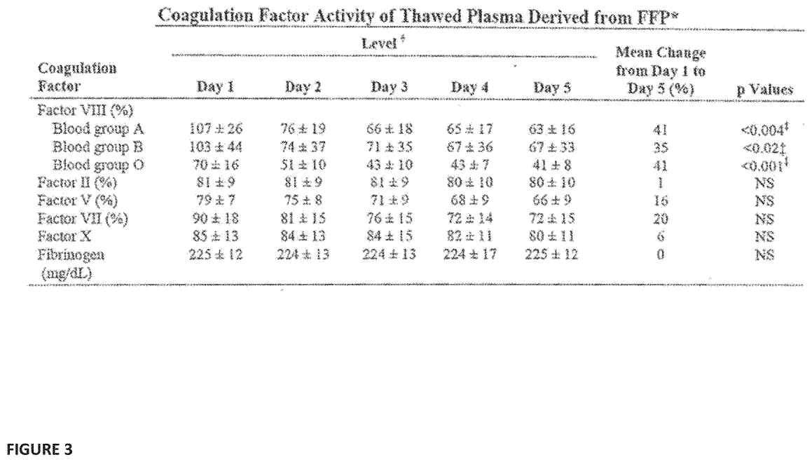

See, FIG. 3.

vWF has generally been difficult to recover and has become one indicator for preservation of all factors. The present invention includes recovering amounts of active/undenatured vWF, in an amount in physiologically active reconstituted spray dried plasma, prior to fractionation, that is at least about 60%, about 70%, at least about 80%, about 90%, or more when compared with the amount of active/undenatured vWF in native plasma. vWF activity is typically assayed with an assay called the von Willebrand factor: Ristocetin cofactor [vWF:RCo] assay, as is known to those of skill in the art. The vWF:RCo assay measures the ability of a patient's plasma to agglutinate platelets in the presence of the antibiotic Ristocetin. The rate of Ristocetin induced agglutination is related to the concentration and functional activity of the plasma von Willebrand factor. Another assay, the vWF antigen assay, measures the amount of vWF protein present in a sample.

In some embodiments, the physiological reconstituted spray dried plasma contains albumin in an amount from about 3.5 to about 5.5 g/dL. In various embodiments, the albumin concentration of the physiologically active reconstituted spray dried plasma is from about 40% to about 70%, e.g., from about 50% to about 60% of the total plasma protein content of the physiologically active reconstituted spray dried plasma.

In various embodiments, the albumin in the physiologically active reconstituted spray dried plasma retains at least about 80%, 85%, 90%, or at least about 95% of the activity on a per unit basis of albumin in plasma.

In some embodiments, the physiological reconstituted spray dried plasma contains A1PI in an amount from about 50-300 mg/dL, e.g., from about 100 to about 200 mg/dL.

In various embodiments, the A1PI in the physiologically active reconstituted spray dried plasma retains at least about 80%, 85%, 90%, or at least about 95% of the activity on a per unit basis of A1PI in plasma.

In various embodiments, the physiological reconstituted spray dried plasma contains IgG in an amount of from about 500 to about 1600 mg/dL, e.g., from about 700 to about 1500 mg/dL.

In various embodiments, the IgG in the physiologically active reconstituted spray dried plasma retains at least about 80%, 85%, 90%, or at least about 95% of the activity on a per unit basis of IgG in plasma.

In some embodiments, the physiologically active spray dried plasma has an average particle size of about 30 microns or less. In some embodiments, the physiologically active spray dried plasma has a maximum particle size of about 100 microns or less.

In some embodiments, the physiologically active reconstituted plasma includes at least 30% plasma protein by weight.

In some embodiments, when reconstituted with 1 mL of fluid per 0.09 grams of powder, the physiologically active reconstituted plasma has a protein concentration in the range of 35 mg/mL to 60 mg/mL.

In some embodiments, the physiologically active reconstituted plasma is sterile.

Following reconstitution of the physiologically active spray dried plasma, the resulting solution is submitted to fractionation. An exemplary mode of fractionation is Cohn fractionation, and its variations.

In an exemplary embodiment, the invention provides a method of fractionating physiologically active reconstituted spray dried human plasma using the Cohn fractionation procedure, for example, that procedure set forth in U.S. Pat. No. 2,390,074, wherein the instant improvement comprises the use of physiologically active reconstituted spray dried human plasma as the starting material for the fractionation procedure. FIG. 1 provides an exemplary process diagram for a method of Cohn fractionation.

Thus, for example, the physiologically active spray dried reconstituted plasma is submitted to a method of fractionating proteins by precipitation from a solution containing a plurality of protein fractions, the solution having a pH above the iso-electric point of the fraction desired to be precipitated, which comprises lowering the pH of the solution to bring the same to approximately the iso-electric point of the desired fraction to be precipitated, bringing the ionic strength of the solution to between 0.1 and 0.2, lowering the temperature of the solution to between approximately 0° C. and the freezing point of the solution, adding an organic precipitation for the protein to the protein solution, the amount of the precipitant added being such as to cause precipitation of the desired fraction only from the protein solution the said temperature, and separating the precipitate from the solution.

In various embodiments, there is provided a method of fractionating proteins by precipitation from a solution of physiologically active reconstituted human plasma containing a plurality of protein fractions, comprises bringing the pH of the solution to approximately the iso-electric point of the desired protein fraction to be precipitated, bring the ionic strength of the solution to between 0.01 and 0.2, lowering the temperature of the solution to between approximately 0° C., and the freezing point of the solution, adding and organic precipitant for protein to the protein solution, the amount of the precipitant added, the pH, the ionic strength and the temperature being such as to cause precipitation of only the desired fraction from the protein solution, and separating the precipitate from the solution.

In various embodiments, in the method for fractionating proteins from a solution of reconstituted physiologically active human plasma, the steps which comprise mixing with a solution of proteins an organic precipitant for protein, adjusting the temperature between 0 and −15° C., the amount of the precipitant between 10% and 40%, the pH between 4.4 and 7 and the ionic strength between 0.05 and 0.2, and separating from the resulting liquid system a protein precipitated which is insoluble therein.

In some embodiments, in the method for fractionating proteins from a solution of reconstituted physiologically active human plasma, the steps which comprise mixing with a solution of proteins an organic precipitant for protein, adjusting and maintaining the temperature above the freezing point thereof but not above 0° C., the amount of the precipitant between 10% and 40%, the pH between 4.4 and 7 and the ionic strength between 0.05 and 0.2, and separating from the resulting liquid system a protein precipitated which is insoluble therein.

In some embodiments, in the method for fractionating proteins from a solution of reconstituted human plasma, the steps which comprise adding to a containing a mixture of proteins, both an electrolyte and an organic precipitant for protein, the electrolyte being added in an amount sufficient to bring the ionic strength to between 0.01 and 0.2, and the precipitant being added in amount such as to cause precipitation of only the desired protein fraction, adjusting and maintaining the pH of the solution between 4.4 and 7 and the temperature thereof between 0 and −15° C., and thereby precipitating a protein from the resulting system.

In an exemplary embodiment, the invention provides a method of purifying and crystallizing albumin from a solution of reconstituted human plasma, which comprises dissolving impure albumin in an alcohol solution containing from 15 to 40% alcohol, at a pH of approximately 5.5 to 6.0, an ionic strength of 0.05 to 0.5 and at a temperature of 0° C. to −5° C., and maintaining said solution within said temperature range until a purified albumin crystallizes out.

In an exemplary embodiment, in a method of fractionating substances which have differing solubilities from a solution of reconstituted human plasma at a controlled temperature and hydrogen ion concentration, removing the precipitate thus formed and precipitating a plurality of successive fractions of said substances by variation in one or more of the factors.

The method of preventing denaturation of proteins by modifying reagents which would normally result in denaturation, which comprises adding the reagents to a protein solution of reconstituted human plasma by diffusion through a semi-permeable membrane.

In one embodiment, there is provided a method for fractionating proteins from a solution of physiologically active reconstituted human plasma comprising contacting the physiologically active reconstituted human plasma with an organic precipitant. An exemplary embodiment includes controlling one or more of the amount of the precipitant in the solution, the temperature, the hydrogen ion concentration and the ionic strength, separating the resulting precipitate from the protein solution, and separating successive protein fractions by varying a plurality of said factors affecting solubility thereof.

In an exemplary embodiment, the organic precipitant is added a temperature of 0° or less than 0° C.

In an exemplary embodiment, the organic precipitant is an alcohol. In various embodiments, it is added a temperature of 0° or less than 0° C.

In an exemplary embodiment, there is provided the method of fractionating proteins from a solution of physiologically active reconstituted human plasma which comprises as steps precipitating a plurality of different protein fractions from the plasma by the plasma with the organic precipitant and by varying the temperature of said plasma, the temperature being progressively lowered and the alcohol concentration of the plasma being increased, with the precipitation of successive protein fractions, the temperature and the percentage of alcohol being so correlated that the temperature employed for the precipitation of any given protein fraction is close to but above the freezing point of the plasma at the percentage of alcohol present therein.

Exemplary organic precipitants include ethanol, acetone, dioxane and combinations thereof.

In an exemplary embodiment, a protein in Cryopaste isolated from the physiologically active reconstituted spray dried human plasma selected from Factor VIII, Factor IX and a combination thereof is isolated in a yield of not less than 80% of the yield in which this protein is isolated from fresh frozen plasma. In various embodiments, the activity of the protein is not less than 60%, 65%, 70%, 75%, 80%, 85%, 90%, or 95% of the activity of the protein isolated from fresh frozen plasma.

In an exemplary embodiment, IgG isolated from the physiologically active reconstituted spray dried human plasma is isolated in a yield of not less than 60%, 65%, 70%, 75%, 80%, 85%, 90%, or 95% of the yield in which this protein is isolated from fresh frozen plasma. In various embodiments, the activity of the IgG is not less than 60%, 65%, 70%, 75%, 80%, 85%, 90%, or 95% of the activity of the IgG isolated from fresh frozen plasma.

In an exemplary embodiment, a protein isolated from Fraction IV-1 of the fractionated physiologically active reconstituted spray dried human plasma selected from A1PI, AT-III and a combination thereof is isolated in a yield of not less than 60%, 65%, 70%, 75%, 80%, 85%, 90%, or 95% of the yield in which this protein is isolated from fresh frozen plasma. In various embodiments, the protein isolated from the physiologically active reconstituted spray dried human plasma in Fraction IV-1 has an activity of not less than 60%, 65%, 70%, 75%, 80%, 85%, 90%, or 95% of the activity of the protein isolated from fresh frozen plasma.

In some embodiments, the invention provides a method wherein albumin isolated from Fraction V of the physiologically active reconstituted spray dried human plasma is isolated in a yield of not less than 80% of the yield in which this protein is isolated from fresh frozen plasma. In various embodiments, the albumin isolated from the physiologically active reconstituted spray dried human plasma has an activity of not less than 60%, 65%, 70%, 75%, 80%, 85%, 90%, or 95% of the activity of albumin isolated from fresh frozen plasma.

The methods provided herein allow for the preparation of A1PI compositions having very high levels of purity. For example, in one embodiment, at least about 95% of the total protein in an A1PI composition provided herein is A1PI. In other embodiments, at least about 96% of the protein in this composition is A1PI, or at least about 97%, 98%, 99%, 99.5%, or more of the total protein of the composition is A1PI.

Similarly, the methods provided herein allow for the preparation of A1PI compositions containing extremely low levels of contaminating agents. For example, in certain embodiments, A1PI compositions are provided that contain less than about 10 mg/L contaminant. In other embodiments, the A1PI composition will contain less than about 5 mg/L contaminant, preferably less than about 3 mg/L contaminant, most preferably less than about 2 mg/L contaminant.

In various embodiments, the A1PI in the physiologically active reconstituted spray dried plasma retains at least about 80%, 85%, 90%, or at least about 95% of the activity on a per unit basis of A1PI in plasma.

In one embodiment, the present invention provides aqueous IgG compositions comprising a protein concentration of between about 150 g/L and about 250 g/L. In certain embodiments, the protein concentration of the IgG composition is between about 175 g/L and about 225 g/L, or between about 200 g/L and about 225 g/L, or any suitable concentration within these ranges, for example at or about, 150 g/L, 155 g/L, 160 g/L, 165 g/L, 170 g/L, 175 g/L, 180 g/L, 185 g/L, 190 g/L, 195 g/L, 200 g/L, 205 g/L, 210 g/L, 215 g/L, 220 g/L, 225 g/L, 230 g/L, 235 g/L, 240 g/L, 245 g/L, 250 g/L, or higher. In a preferred embodiment, the aqueous IgG composition comprises a protein concentration of at or about 200 g/L. In a particularly preferred embodiment, the aqueous IgG composition comprises a protein concentration of at or about 204 g/L.

The methods provided herein allow for the preparation of IgG compositions having very high levels of purity. For example, in one embodiment, at least about 95% of the total protein in an IgG composition provided herein will be IgG. In other embodiments, at least about 96% of the protein is IgG, or at least about 97%, 98%, 99%, 99.5%, or more of the total protein of the composition will be IgG.

Similarly, the methods provided herein allow for the preparation of IgG compositions containing extremely low levels of contaminating agents. For example, in certain embodiments, IgG compositions are provided that contain less than about 100 mg/L IgA. In other embodiments, the IgG composition will contain less than about 50 mg/L IgA, preferably less than about 35 mg/L IgA, most preferably less than about 20 mg/L IgA.

In some embodiments, the invention provides a preparation of polyvalent and/or hyperimmune immunoglobulins (IgGs) prepared by a method of the invention. In various embodiments, the IgG is selected from anti-RhO hyperimmune immunoglobulin, anti-hepatitis B hyperimmune immunoglobulin, anti-rabies hyperimmune immunoglobulin, anti-tetanus IgG hyperimmune immunoglobulin and a combination of any two or more thereof

In various embodiments, the IgG in the physiologically active reconstituted spray dried plasma retains at least about 80%, 85%, 90%, or at least about 95% of the activity on a per unit basis of IgG in plasma.

### Spray Dryer and the Spray Drying Process

The physiologically active dried plasma, which is reconstituted and subsequently fractionated is dried by spray drying in a spray dryer system. In general, a spray dryer system (spray dryer device) is provided for spray drying a liquid sample such as blood plasma. In an embodiment, the spray dryer system used to spray dry plasma for reconstitution by the solution of the present disclosure includes a spray dryer device and a spray dryer assembly. The spray dryer device is adapted, in an aspect, to receive flows of an aerosolizing gas, a drying gas, and plasma liquid from respective sources and coupled with the spray dryer assembly. The spray dryer device can further transmit the received aerosolizing gas, drying gas, and plasma to the spray dryer assembly. Spray drying of the plasma is performed in the spray dryer assembly under the control of the spray dryer device. Any suitable spray drying system can be used to dry plasma for use in with present invention. For exemplification, a suitable spray dryer is described below.

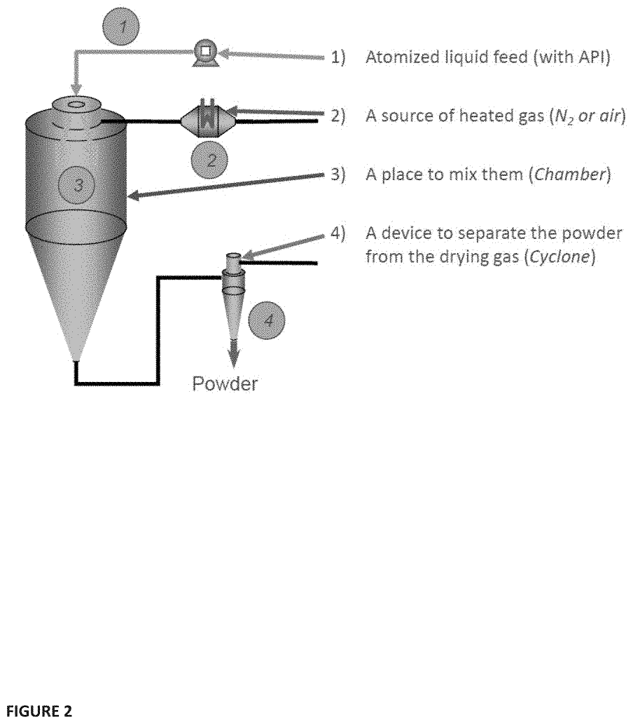

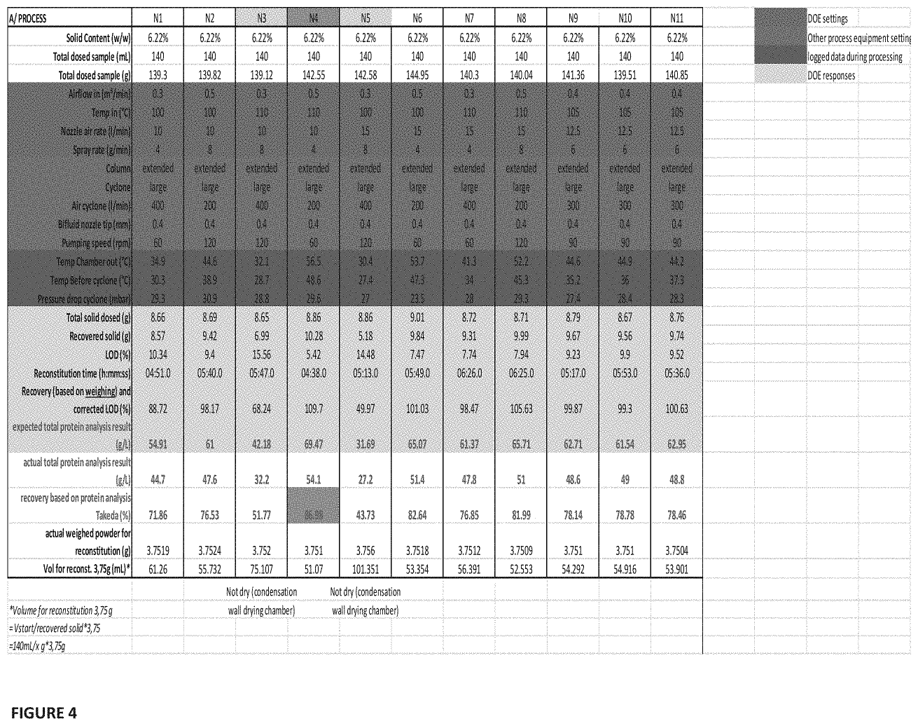

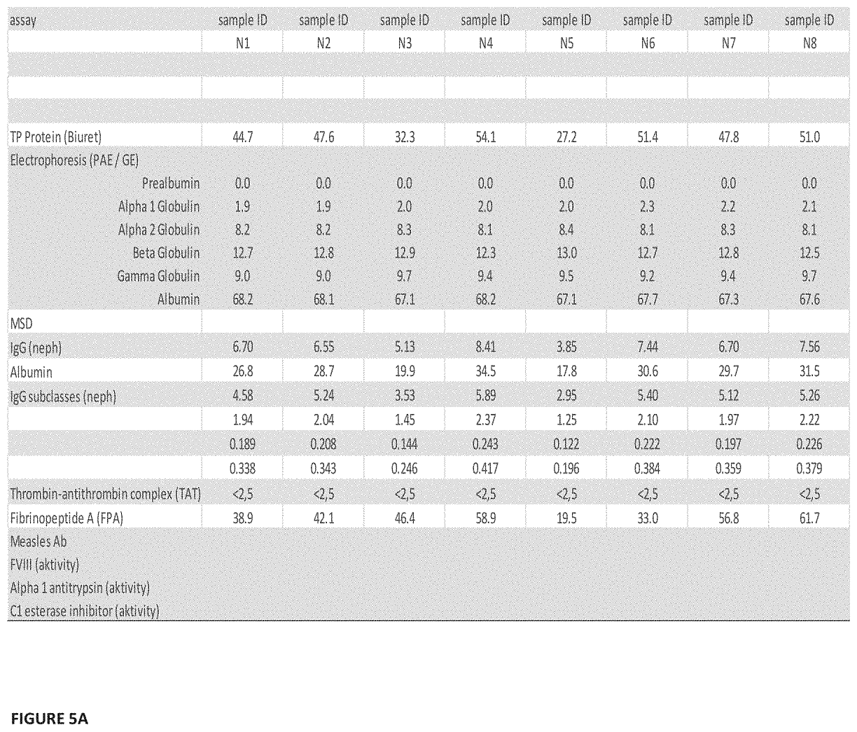

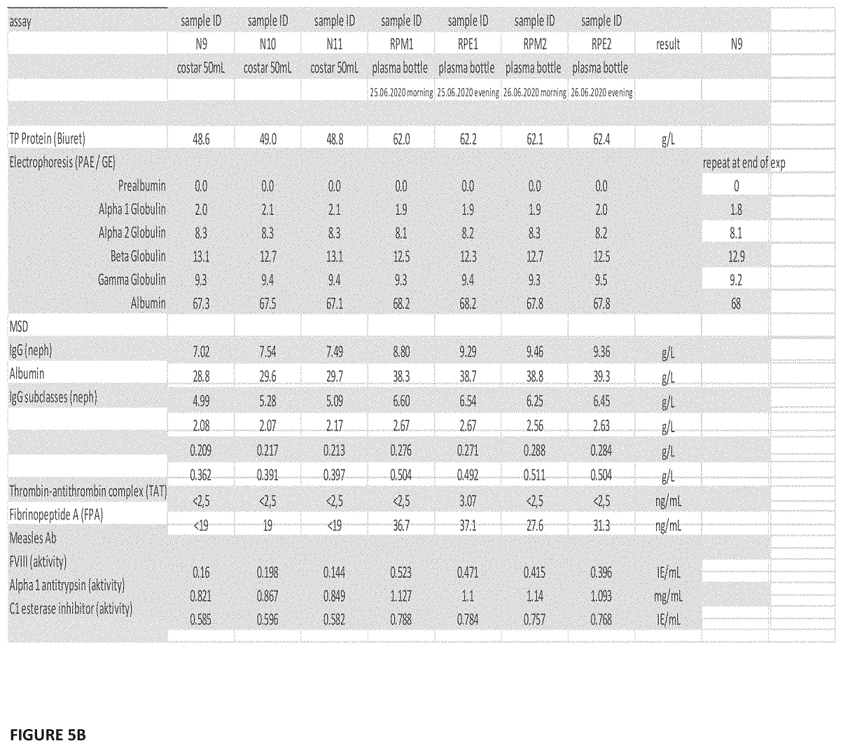

An exemplary spray drying apparatus of use in the invention is provided in FIG. 2. Exemplary spray drying process parameters are provided in FIG. 4.

In certain embodiments, the spray dryer assembly includes a sterile, hermetically sealed enclosure body and a frame to which the enclosure body is attached. The frame defines first, second, and third portions of the assembly, separated by respective transition zones. A drying gas inlet provided within the first portion of the assembly, adjacent to a first end of the enclosure body.

A spray drying head is further attached to the frame within the transition zone between the first and second portions of the assembly. This position also lies within the incipient flow path of the drying gas within the assembly. During spray drying, the spray drying head receives flows of an aerosolizing gas and plasma and aerosolizes the plasma with the aerosolizing gas to form an aerosolized plasma. Drying gas additionally passes through the spray drying head to mix with the aerosolized plasma within the second portion of the assembly for drying. In the second portion of the assembly, which functions as a drying chamber, contact between the aerosolized plasma and the drying gas causes moisture to move from the aerosolized plasma to the drying gas, producing dried plasma and humid drying gas.

In alternative embodiments, the aerosolizing gas can be omitted and the spray dryer assembly head may include an aerosolizer that receives and atomizes the flow of plasma. Examples of the aerosolizer may include, but are not limited to, ultrasonic atomizing transducers, ultrasonic humidified transducers, and piezo-ultrasonic atomizers. Beneficially, such a configuration eliminates the need for an aerosolizing gas, simplifying the design of the spray dryer device and assembly and lowering the cost of the spray dryer system.

The spray drying head in an embodiment is adapted to direct the flow of drying gas within the drying chamber. For example, the spray drying head includes openings separated by fins which receive the flow of drying gas from the drying gas inlet. The orientation of the fins allows the drying gas to be directed in selected flow pathways (e.g., helical). Beneficially, by controlling the flow pathway of the drying gas, the path length over which the drying gas and aerosolized blood plasma are in contact within the drying chamber is increased, reducing the time to dry the plasma.

The physiologically active dried plasma and humid drying gas subsequently flow into the third portion of assembly, which houses a collection chamber. In the collection chamber, the dried plasma is isolated from the humid drying gas and collected using a filter. For example, the filter in an embodiment is open on one side to receive the flow of humid air and dried plasma and closed on the remaining sides. The humid drying gas passes through the filter and is exhausted from the spray dryer assembly.

In alternative embodiments, the filter is adapted to separate the collection chamber into two parts. The first part of the collection chamber is contiguous with the drying chamber and receives the flow of humid drying gas and dried plasma. The dried plasma is collected in this first part of the collection chamber, while the humid air passes through the filter and is exhausted from the spray dryer assembly via an exhaust in fluid communication with the second part of the spray dryer assembly.

After collecting the physiologically active dried plasma, the collection chamber is separated from the spray dryer assembly and hermetically sealed. In this manner, the sealed collection chamber is used to store the dried plasma until use. The collection chamber includes a plurality of ports allowing addition of the reconstitution solution of the present invention to the collection chamber for reconstitution of the blood plasma and removal of the reconstituted blood plasma for use. The collection chamber can further be attached to a sealed vessel containing the reconstitution solution for reconstitution.

When handling transfusion products such as blood plasma, the transfusion products must not be exposed to any contaminants during collection, storage, and transfusion. Accordingly, the spray dryer assembly, in an embodiment, is adapted for reversible coupling with the spray dryer device. For example, the spray dryer assembly is coupled to the spray dryer device at about the drying gas inlet. Beneficially, so configured, the spray dryer assembly accommodates repeated or single use. For example, in one embodiment, the spray dryer assembly and spray drying head is formed from autoclavable materials (e.g., antibacterial steels, antibacterial alloys, etc.) that are sterilized prior to each spray drying operation. In an alternative embodiment, the spray dryer head and spray drying chamber is formed from disposable materials (e.g., polymers) that are autoclaved prior to each spray drying operation and disposed of after each spray drying operation.

Apparatuses and methods for spray drying are known in art. Spray drying methods and apparatus are further described in U.S. Pat. Nos. 8,469,202, 8,533,971, 8,407,912, 8,595,950, 8,601,712, 8,533,972, 8,434,242, US Patent Publication Nos. 2016/0082044, 20160084572, 2010/0108183, 2011/0142885, 2013/0000774, 2013/0126101, 2014/0083627, 2014/0083628, and 2014/0088768, the entire teachings of which are incorporated herein by reference for all purposes.

The following Examples are offered to illustrate exemplary embodiments of the invention and do not define or limit its scope.

### EXAMPLES

### Example 1

The complete process of spray drying involves a sequence of four processes. The dispersion is achieved with a pressure nozzle, a two fluid nozzle, a rotary disk atomizer or an ultrasonic nozzle. Selection of the atomizer type depends upon the nature and amount of feed and the desired characteristics of the dried product. The higher the energy for the dispersion, the smaller are the generated droplets. The manner in which spray contacts the drying air is an important factor in spray drying, as this has great bearing on dried product properties by influencing droplet behavior during drying. In one example, the material is sprayed in the same direction as the flow of hot air through the apparatus. The droplets come into contact with the hot drying gas when they are the most moist. In another example, the material is sprayed in the opposite direction of the flow of hot gas. The hot gas flows upwards and the product falls through increasingly hot air into the collection tray. The residual moisture is eliminated, and the product becomes very hot. This method is suitable only for thermally stabile products. In yet another embodiment, the advantages of both spraying methods are combined. The product is sprayed upwards and only remains in the hot zone for a short time to eliminate the residual moisture. Gravity then pulls the product into the cooler zone. This embodiment is particularly advantageous because the product is only in the hot zone for a short time, and is less likely to be affected by heat.

In the spray drying method, air is mostly used as drying medium, but other gases such as nitrogen may also be used. The gas stream is heated electrically or in a burner and after the process it is exhausted to atmosphere. If the heating medium is recycled and reused, typically an inert gas such as nitrogen, is used instead of air. Use of nitrogen is advantageous when flammable solvents, toxic products or oxygen sensitive products are processed.

During the spray drying process, as soon as droplets of the spray come into contact with the drying gas, evaporation takes place from the saturated vapor film which is quickly established at the droplet surface. Due to the high specific surface area and the existing temperature and moisture gradients, heat and mass transfer results in efficient drying. The evaporation leads to a cooling of the droplet and thus to a small thermal load. Drying chamber design and air flow rate provide a droplet residence time in the chamber, so that the desired droplet moisture removal is completed and product removed from the dryer before product temperatures can rise to the outlet drying air temperature. Hence, there is little likelihood of heat damage to the product.

Two systems are used to separate the product from the drying medium. First, primary separation of the drying product takes place at the base of the drying chamber, and second, total recovery of the dried product in the separation equipment. In one embodiment, a cyclone is used to collect the material. Based on inertial forces, the particles are separated to the cyclone wall as a down-going strain and removed. Other systems such as electrostatic precipitators, textile (bag) filters or wet collectors like scrubbers, may also be used to collect the dried product.

As used in the present invention, spray drying offers advantages over other drying methods such as lyophilization (freeze drying). Use of spray drying produces a product that is more consistent, less clumpy, and better dispersed than freeze drying methods. The highly dispersed particles produced by spray drying also allow for a rapid rehydration rate, which is likely a result of a larger available surface area. By contrast, the clumped nature of a freeze dried product, results in substantially longer rehydration times for the blood products that are dried in the method of the invention. Since many transfusions and other uses of blood products can be highly time-sensitive, this higher rate of rehydration can be a significant advantage in battlefield or emergency treatment situations. As explained in more detail below, spray dried fixed blood platelets of the invention can be rehydrated to form a rehydrated fixed blood platelet composition, and the composition has a turbidity (A.sub.500) value less than that of a comparable rehydrated lyophilized composition of fixed blood platelets.

### Example 2

| | |
|---|---|
| 1. | Spray-drying equipment to be used |

4M8-Trix spray dryer (ProCepT, Zelzate, Belgium)
Dimensions of the drying chamber:

Straight drying chamber: height 60 cm, dm 18.4 cm

1 or 2 levels of straight drying chamber

Conical drying chamber: height 75 cm, dm 18.4 cm
Total length of drying chamber: ±135 cm-195 cm

Two-fluid nozzle

Fluid enters at the top of the spray dryer by a 12 roller peristaltic pump with a
Tygon ® MHLL tube (inside diameter: 1.14 mm or 2.79 mm) with an
Isamprene outer coating

Co-current airflow
Collection of powder in a reservoir attached to the cyclone
Water evaporation capacity: Max. 3 L/h
Process parameters

Airflow: 0.2 m³/min-1 m³/min
Temperature in (° C.): Max 200° C.
Bifluid nozzle tip (mm): 0.2-0.4-0.6-0.8-1.0-1.2 mm
Air/Liquid ratio:

Nozzle air rate (L/min): Max. 25 L/min
Spray rate (g/min): 0.1-15 g/min

| | |
|---|---|
| 2. | Experimental |
| | 60 L of frozen plasma is stored at −20° C. |

### a. Plasma Pre-Treatment Prior to Spray Drying

After taking the plasma bags containing plasma to be spray dried from the freezer (−20° C.), the plasma bags are rapidly thawed to 28-30° C. using a water bath. Next, the thawed plasma is pooled. Pooled plasma is stored at 8° C. with continuous stirring. Pooled plasma required can remain at 5-8° C. for 3 days. The amount of plasma from the pool needed for the infeed of a spray drying run is brought to 28° C. using a water bath and gently stirred during spray drying, assuring there is no foaming. The plasma has a viscosity comparable to fresh plasma.

Viscosity of the plasma pool is determined using a Haake Mars III rheometer (Thermo Scientific, MA, USA). Also turbidity of the plasma pool is measured. Viscosity and turbidity of the plasma pool are measured at 28° C.

### b. Spray-Drying

Phase 1 spray drying is divided into several consecutive protocols as indicated in Table 1. 30 L (out of the 60 L) total is used for protocols 1 and 1.5.

TABLE 1
Process factors and their ranges

| Factor | Range |
|---|---|
| Airflow in | 0.3-0.5 |
| (m³/min) | |
| Temp in | 100-110 |
| (° C.) | |
| Nozzle air | 10-15 |
| rate (l/min) | |
| Spray rate | 4-8 |
| (g/min) | |

In this Example, the required amount of plasma for the spray-drying was assembled by pooling (as outlined above), ensuring that homogeneous plasma is used for the entire protocol. The spray drying process parameters and their ranges within which they are varied using a 2-level fractional factorial approach (i.e., 2⁴⁻¹+3 centre point experiments=11 experiments are listed in the following Table 2:

TABLE 2
Overview 2-level fractional factorial design experiments

| Exp | Exp | Run | Incl/ | airflow | temper- | nozzle air | spray |
|---|---|---|---|---|---|---|---|
| No | Name | Order | Excl | in | ature in | rate | rate |
| 1 | N1 | 6 | Incl | 0.3 | 100 | 10 | 4 |
| 2 | N2 | 3 | Incl | 0.5 | 100 | 10 | 8 |
| 3 | N3 | 4 | Incl | 0.3 | 110 | 10 | 8 |
| 4 | N4 | 8 | Incl | 0.5 | 110 | 10 | 4 |
| 5 | N5 | 7 | Incl | 0.3 | 100 | 15 | 8 |
| 6 | N6 | 9 | Incl | 0.5 | 100 | 15 | 4 |
| 7 | N7 | 10 | Incl | 0.3 | 110 | 15 | 4 |
| 8 | N8 | 2 | Incl | 0.5 | 110 | 15 | 8 |
| 9 | N9 | 1 | Incl | 0.4 | 105 | 12.5 | 6 |
| 10 | N10 | 5 | Incl | 0.4 | 105 | 12.5 | 6 |
| 11 | N11 | 11 | Incl | 0.4 | 105 | 12.5 | 6 |

This protocol contains 3 replicate experiments. One replicate was run at the start, one at the middle and one at the end of the experiment, allowing evaluation of a time effect of the pooled plasma.

These process parameter ranges were selected based on literature information^{1,2}. In this literature, plasma was spray dried using a Büchi spray drying system under certain process settings. These settings were translated into process parameter ranges applicable on the 4M8-Trix spray dryer (ProCepT, Zelzate, Belgium) used in this study.

The responses evaluated were: processability, yield, residual moisture content, solubility/re-suspension and the test panel. To measure these responses, 5.25 g of spray dried powder per experiment run was used (i.e.; 3.75 g for the test panel; 1.50 g for the 2 residual moisture measurements).

Taking into account a potential maximum spray drying yield loss 25% and taking into account that 1L of plasma contains 50 g of proteins, this means that 140 mL of plasma is spray dried per experimental run, resulting in at least 5.25 g of spray dried powder (75% (140 ml (50 g/1000 ml)=5.25 g). With 11 experimental runs, a plasma pool of approx. 1600 mL is utilized for the spray drying experiment. In addition, 140 mL pooled plasma is separated per day prior to spray drying for reference analysis (i.e., approx. 280 mL of pooled plasma in total for reference analysis). A plasma pool of approx. 1900 mL is prepared (i.e., 3 plasma bags). The spray drying of 11 runs (140 ml pooled plasma per run) takes 2 days (i.e., approx. 1 hour/run).

FIG. 3 provides detail for an exemplary spray drying run and data on reconstitution and the properties of the spray dried plasma and reconstituted plasma.

### c. References

¹ https://www.ncbi.nlm.nih.gov/pmc/articles/PMC3891503/
² http://patft.uspto.gov/netacgi/nph-Parser?Sect1=PTO2&Sect2=HITOFF&p=1&u=%2Fnetahtml%2FPTO%2Fsearch-adv.htm&r=4&f=G&1=50&d=PTXT&S1=%22dried+human+plasma%22&OS=%22dried+human+plasma%22&RS=%22dried+human+plasma%22

### d. Spray Dried Powder Evaluation

For each spray drying experiment, the processability, yield and residual moisture content of the spray dried powder is analyzed. The remainder of the spray dried powder is submitted to reconstitution and characterization by methods, which are generally recognized in the art. FIG. 3. Characterization of the spray dried plasma according to various art-recognized standards provided the results shown in FIG. 4A, and FIG. 4B.

Notably, the spray dried plasms (PptG) paste is comparable in color and texture to PptG paste from the control. The process demonstrated comparable IgG recovery at II+III extract vs. control. The spray dried plasma showed reasonable precipitate ratio when suspended at 28° C. or 1° C. The control samples and the spray dried suspensions showed similar fibrinogen results before and after centrifugation. The spray dried suspensions showed significantly lower turbidity values than the control. All conditions showed similar IgG results before and after centrifugation.

### Example 3

This example provides conditions for an exemplary process of the invention, such as the process set forth in FIG. 6.

### 3.1 Materials and Methods

| Step | Parameters | Run | Control | Test 1 | Test 2 |
|---|---|---|---|---|---|
| CRP to Fr. | Plasma Source | | Frozen | F | |
| I @ 8% | Lot Number | | | | |
| | Plasma volume needed | L | | 7.5 | 6 |
| | Amount of Plasma powder needed | g | | 615 | 492 |
| | Weight of water needed | kg | | 6.885 | 5.508 |
| | Actual water needed | L | | | 4 |
| | Actual water used | L | | | 4.15 |
| | pH of suspension | | | 9.46 | 9.51 |
| | conductivity of suspension | mS/cm | | 11.972 | 14.1 |
| | turbidity of suspension | NTU | | 308 | 331 |
| | Weight of supernatant | kg | | 7.398 | |
| | Weight of precipitant | kg | | 0.029 | |
| | pH of supernatant | | | 9.45 | |
| | conductivity of supernatant | mS/cm | | 12 | |
| | turbidity of supernatant | NTU | | 280 | |
| | precipitant to supernatant ratio | g/kg CPP | | 3.92 | |
| | Pooled weight of Plasma (CRP) | kg | 5.836 | 7.500 | 4.642 |
| | Volume of Plasma (CRP) | L | 5.6881 | 7.3099 | 4.5244 |
| | Weight of CPP after Centrifugation | kg | 5.411 | 7.398 | 0 |
| | Volume of CPP after Centrifugation | L | 5.2790 | 7.2176 | 0.0000 |
| | Weight Cryo Precipitate | kg | 0.0773 | | |
| | Cryo Precipitate yield | g/L CPP | 14.6429 | | |
| | Quantity of CPP (volume) at start | L | 5.00 | 5.00 | 4.5 |
| | Quantity of CPP (weight) | kg | 5.125 | 5.125 | 4.613 |
| | Bulk Temp | ° C. | 1.6 | 3.3 | 1.2 |
| | Initial pH | | 8.21 | 9.61 | 9.51 |
| | Final pH (target 7.20) | | 7.20 | 7.21 | 7.14 |
| | Amt of diluted pH 4.0 buffer | mL | 2.09 | 4.4 | 2.75 |
| | Amount of pH 4.0 Buffer | L | 0.010 | 0.022 | 0.025 |
| | Weight of pH 4.0 Buffer | kg | 0.011 | 0.023 | 0.026 |
| | Sample removed for pH | mL | 10 | 10 | 10 |
| | pH check | | 7.30 | 7.40 | 7.22 |
| | Total Volume of Bulk | L | 5.010 | 5.022 | 4.525 |
| | Amt of 95% Alcohol Required | kg | 0.371 | 0.372 | 0.335 |
| | Volume of 95% Alcohol | L | 0.446 | 0.447 | 0.402 |
| | Total Calculated Bulk Weight | kg | 5.507 | 5.520 | 4.974 |
| | Total Calculated Bulk Volume | L | 5.456 | 5.469 | 4.927 |
| | Sample removed for pH | mL | 10 | 10 | 10 |
| | pH after alcohol add initial | | 7.48 | 7.56 | 7.4 |
| | pH after alcohol add adjusted | | 7.09 | 7.05 | 7.08 |
| | Amt of diluted pH 4.0 buffer | mL | 0.08 | 0.096 | 0.05 |
| | Amount of pH 4.0 Buffer | L | 0.004 | 0.005 | 0.002 |
| | Weight of pH 4.0 Buffer | kg | 0.005 | 0.006 | 0.003 |
| | Sample removed for pH | mL | 10 | 10 | 10 |
| | pH check before overnight aging | | 7.18 | 7.09 | 7.21 |
| | Total Aging Time | hours | 15.88 | 16.92 | 18.65 |
| | pH check after overnight aging | | 7.58 | 7.36 | 7.52 |
| | Sample removed for pH | | 10 | 10 | 10 |
| | Adjusted pH | | 7.16 | | 7.16 |
| | Amt of diluted pH 4.0 buffer | mL | 0.1 | | 0.07 |
| | Amount of pH 4.0 Buffer | L | 0.0055 | 0.0000 | 0.0035 |
| | Weight of pH 4.0 Buffer | kg | 0.0058 | 0.0000 | 0.0037 |
| | final pH (target 7.20) | | 7.18 | | 7.23 |
| | Sample removed for pH | mL | 10 | | 10 |
| | Suspension weight | kg | 5.52 | 5.53 | 4.98 |
| | Suspension volume | L | 5.57 | 5.58 | 5.03 |
| | Weight of Fr I Centrifugate | kg | 5.3331 | 5.2400 | 4.6533 |
| | Volume of Fr I Centrifugate | L | 5.3870 | 5.2929 | 4.7003 |
| | sampling volume | L | 0.0500 | 0.0500 | 0.0500 |
| Fr I Cent to | Weight of Fr I Precipitate | kg | 0.0813 | 0.1400 | 0.1113 |
| Fr II + III | Initial pH | | 7.21 | 7.38 | 7.24 |
| @ 20% Filt | Final pH (target 6.70) | | 6.70 | 6.70 | 6.72 |
| | Amt of diluted pH 4.0 buffer | mL | 1.7 | 1.5 | 1.48 |
| | Amount of pH 4.0 Buffer | L | 0.009 | 0.008 | 0.007 |
| | Weight of pH 4.0 Buffer | kg | 0.010 | 0.008 | 0.007 |
| | Sample removed for pH | mL | 100 | 100 | 100 |
| | pH check | | 6.68 | 6.75 | 6.72 |
| | Sample removed for pH | mL | 10.00 | 10.00 | 10.00 |
| | Total Volume of Bulk | L | 5.396 | 5.301 | 4.707 |
| | Amt of 95% Alcohol Required | kg | 0.718 | 0.705 | 0.626 |
| | Volume of 95% Alcohol | L | 0.863 | 0.847 | 0.752 |
| | Total Calculated Bulk Weight | kg | 6.060 | 5.953 | 5.287 |
| | Total Calculated Bulk Volume | L | 6.184 | 6.075 | 5.395 |
| | Sample removed for pH | mL | 10 | 10 | 10 |
| | pH after alcohol add | | 7.17 | 7.12 | 6.93 |
| | pH adj before Aging | | 6.9 | 6.89 | 6.93 |
| | Amt of diluted pH 4.0 buffer | mL | 0.06 | 0.035 | 0 |
| | Amount of pH 4.0 Buffer | L | 0.004 | 0.002 | 0.000 |
| | Weight of pH 4.0 Buffer | kg | 0.004 | 0.002 | 0.000 |
| | Sample removed for pH | mL | 10 | 10 | |
| | pH check before overnight aging | | 6.90 | 6.94 | 6.93 |
| | Total Aging Time | hours | 15.97 | 16.93 | 16.93 |
| | pH check after overnight aging | | 7.00 | 7.05 | 7.04 |
| | Sample removed for pH | | 10 | 10 | 10 |
| | Adjusted pH | | | | |
| | Amt of diluted pH 4.0 buffer | mL | | | |
| | Amount of pH 4.0 Buffer | L | 0.0000 | 0.0000 | 0.0000 |
| | Weight of pH 4.0 Buffer | kg | 0.0000 | 0.0000 | 0.0000 |
| | final pH (target 7.00) | | | | |
| | Fr II + III @ 20% Susp Weight | kg | 6.064 | 5.956 | 5.287 |
| Fr. II + | Fr II + III @ 20% Susp Volume | L | 6.126 | 6.016 | 5.340 |
| III | Filtrate pooled Alcohol concentration | % v/v | 16.4 | 16.6 | 20.3 |
| | Weight of Filtrate pooled | kg | 5.25 | 5.0435 | 4.6301 |
| | Volume of Filtrate pooled | L | 5.36 | 5.15 | 4.72 |
| | Turbidity of filtrate pooled | NTU | 9.05 | 10.4 | 16.3 |
| | Weight of Blow Dry | kg | 0.463 | 0.6276 | 0.354 |
| | Weight of Fr. II + III Paste | kg | 0.5032 | 0.477 | 0.543 |
| | Paste Yield | g/L CPP | 100.6 | 95.4 | 90.5 |
| | Fr II + III Paste Weight forward | kg | 0.5032 | 0.477 | 0.543 |
| | Equivalent Plasma Volume | L CPP | 5 | 5 | 6 |
| | Wet Fr II + III Precipitate weight ratio | kg/L CPP | 0.0437 | 0.0437 | 0.0437 |
| | Wet Fr II + III Precipitate weight | kg | 0.219 | 0.219 | 0.262 |
| | Amount of Extract buffer required | kg | 4.26 | 4.26 | 5.11 |
| | Weight of suspension before pH adj | kg | 4.76 | 4.74 | 5.66 |
| | pH of suspension | | 5.16 | 5.16 | 5.16 |
| | Amount of diluted buffer | mL | 0.23 | 0.25 | 0.24 |
| | Amount of undiluted buffer | L | 0.0011 | 0.0012 | 0.0014 |
| | Weight of undiluted buffer | kg | 0.0012 | 0.0013 | 0.0014 |
| | Final pH after adjustment (target 5.00-5.05) | | 5.06 | 5.07 | 5.06 |
| | Weight of suspension after pH adj | kg | 4.77 | 4.74 | 5.66 |
| | Extraction time | hrs | 3.37 | 4.33 | 4.05 |
| | sampling volume | L | 0.05 | 0.05 | 0.05 |
| | Weight of CUNO Filtrate pooled | kg | 5.3 | 5 | 5.9671 |
| | Turbidity of Fr II + III Extract Filtrate | NTU | 15.3 | 21.4 | 16.8 |
| | pooled | | | | |
| | Weight of Blow Dry | kg | 0.2216 | 0.599 | 0.5168 |
| | Weight Fr II + III Extract Precipitate | kg | 0.3993 | 0.4077 | 0.4777 |
| | Paste Yield | g/L CPP | 79.9 | 81.5 | 79.6 |
| | Amount of Fr II + III Ext CUNO Filtrate | kg | 5.3 | 5 | 5.9671 |
| | forward | | | | |
| | Equivalent Plasma Volume | L CPP | 5 | 5 | 6 |
| Fr. II + | Weight filtrate bulk prior to pH Adj | kg | 5.353 | 5.050 | 6.027 |
| III Ext CUNO | Initial pH | | 6.34 | 6.34 | 6.36 |
| Filtrate to | Adjusted pH | | 6.9 | 6.9 | 6.85 |
| Ppt G | Amount of diluted 1N NaOH | mL | 5.3 | 5.4 | 5.1 |
| | Amount of 1N NaOH for pH adj | L | 0.028 | 0.027 | 0.031 |
| | Weight of NaOH | kg | 0.030 | 0.028 | 0.032 |
| | Final pH | | 6.91 | 6.93 | 6.85 |
| | Weight of Filtrate Bulk after pH adj | kg | 5.38 | 5.08 | 6.06 |
| | Amt of 95% Alcohol Required | kg | 1.61 | 1.52 | 1.82 |
| | Total Weight G @ 25% | kg | 6.9973 | 6.6019 | 7.8764 |
| | Total Volume G @ 25% | L | 7.15 | 6.75 | 8.05 |
| | Sample removed for pH | | 100.00 | 100.00 | 100.00 |
| | pH after alcohol addition | | 7.20 | 7.25 | 7.16 |
| | Adjusted pH | | | 7 | 7.02 |
| | Amount of diluted pH 4.0 Buffer | mL | | 0.4 | 0.2 |
| | Total Amount of pH 4.0 buffer | L | | 0.0027 | 0.0016 |
| | Weight of pH 4.0 Buffer | kg | | 0.0029 | 0.0017 |
| | Sample removed for pH | | | 10 | 10 |
| | Total volume of G @ 25% after pH adj | L | 7.15 | 7.05 | 7.06 |
| | Aging time | hours | 16.48 | 16.53 | 17.47 |
| | Sample removed for pH | | 10.00 | 10.00 | 10.00 |
| | pH after aging | | 7.24 | 7.16 | 7.04 |
| | Amount of diluted pH 4.0 Buffer | mL | 0.05 | | |
| | Total Amount of pH 4.0 buffer | L | 0.004 | 0.000 | 0.000 |
| | Weight of pH 4.0 Buffer | kg | 0.004 | 0.000 | 0.000 |
| | Final pH before filtration | | 6.97 | 7.16 | 7.04 |
| | Sample removed for pH | mL | 10 | 10 | 10 |
| | Filtration time | minutes | 43 | 47 | 58 |
| | Weight of G Filtrate | kg | 5.707 | 5.189 | 6.479 |
| | Turbidity of G @ 25% | NTU | 10.4 | 15.0 | 6.8 |
| | Weight of Blow Dry | kg | 0.8675 | 0.9166 | 0.7886 |
| | Weight of Ppt G | kg | 0.1451 | 0.1371 | 0.1541 |
| | % Step Recovery | % | 96% | 95% | 94% |
| | Paste Yield | g/L CPP | 29.02 | 27.42 | 25.68 |

### 3.2 Results

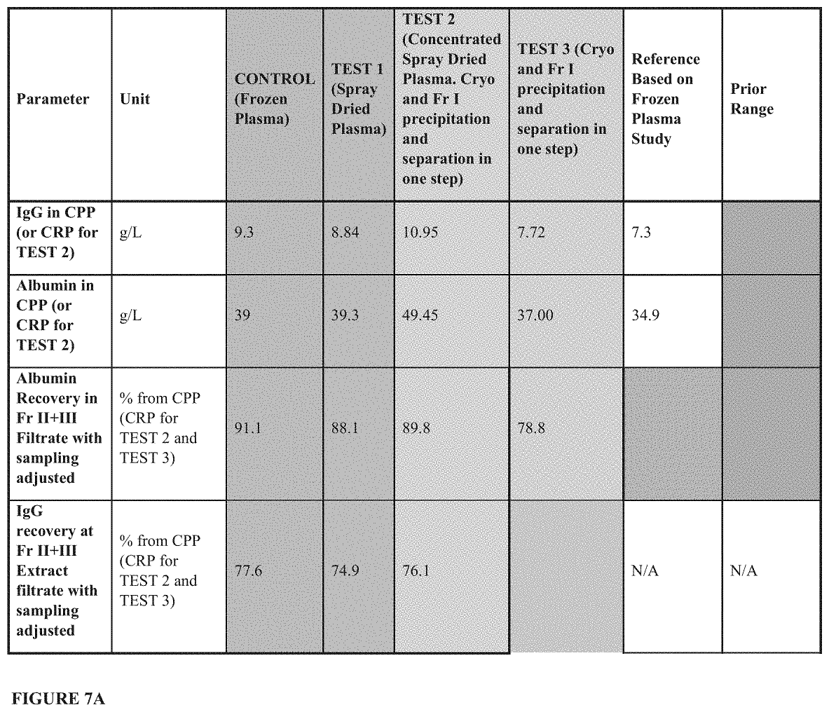

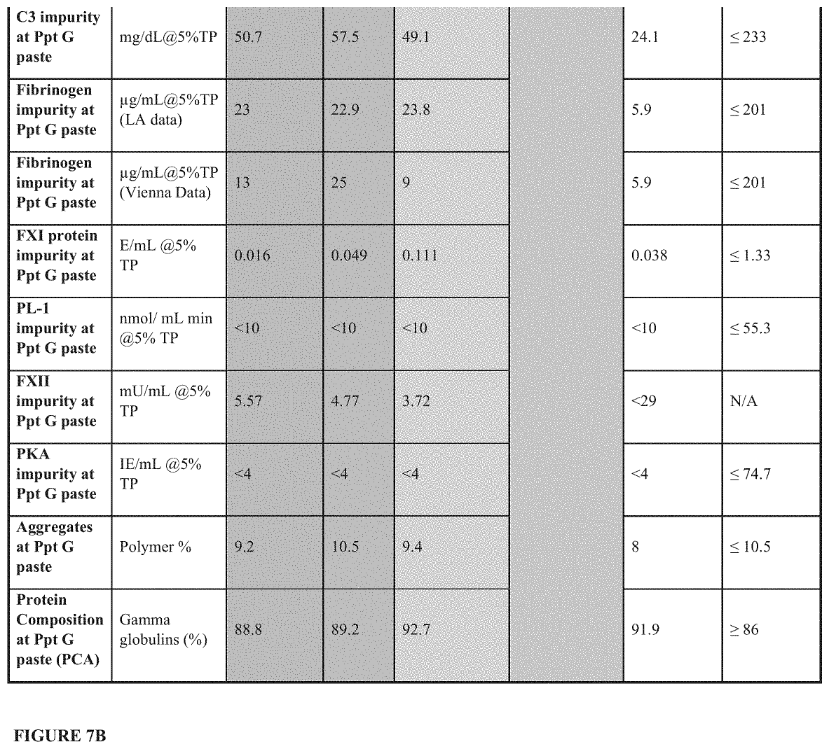

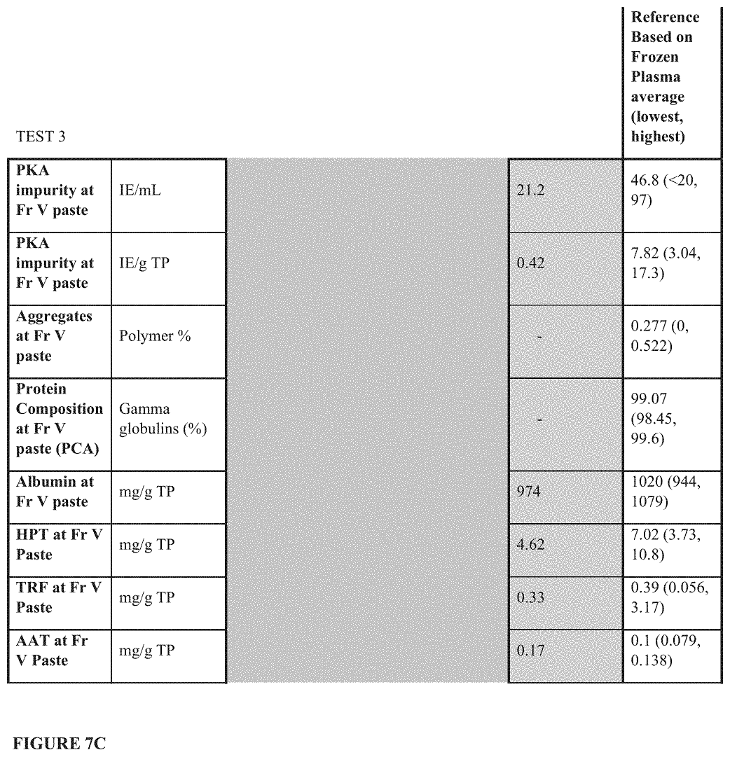

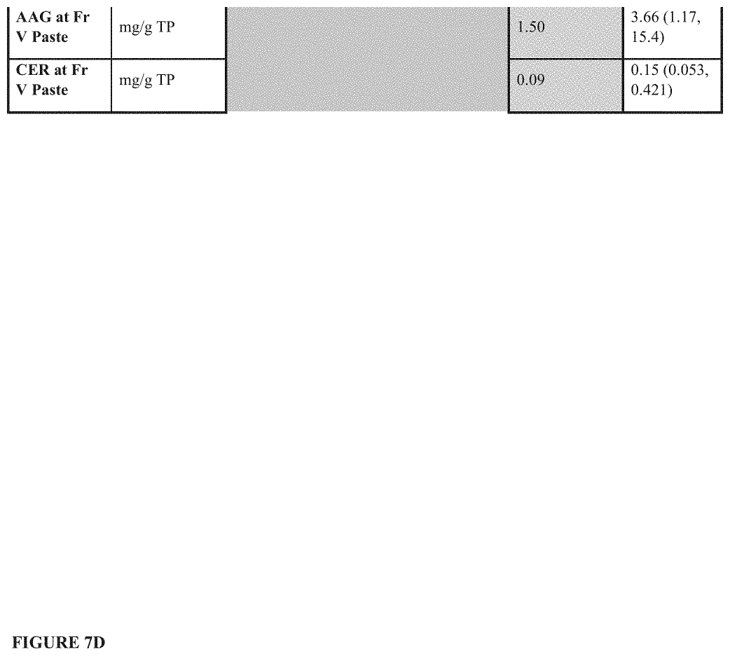

Results from this study are tabulated in FIG. 7A-7D.

In an exemplary experimental run, Fraction I paste recovery for the spray dried plasma was higher than that for the control. This was due to lower recovery of cryo-precipitation (the cryo was carried over to Fraction I precipitation). Spray dried plasma concentrated (approx. 25%) with Cryo and Fr I precipitation and separation in one step (CRP directly to Fr1 step)—Ppt G produced from this test was comparable to control frozen source plasma. This demonstrates that the process has the capacity to combine Cryo and fraction I removal together.

The present invention has been illustrated by reference to various exemplary embodiments and examples. As will be apparent to those of skill in the art other embodiments and variations of this invention may be devised by others skilled in the art without departing from the true spirit and scope of the invention. The appended claims are to be construed to include all such embodiments and equivalent variations.

The disclosures of each and every patent, patent application, and publication cited herein are hereby incorporated herein by reference in their entirety.

What is claimed is:

1. A method of fractionating human plasma using the Cohn. fractionation procedure, wherein the improvement comprises the use of physiologically active reconstituted spray dried human plasma as the starting material for the fractionation procedure.

2. The method according to claim 1, wherein cryopaste is isolated from the physiologically active reconstituted spray dried human plasma and a protein selected from Factor VIII, Factor IX and a combination thereof is isolated from the cryopaste in a yield of not less than 80% of the yield in which this protein is isolated from fresh frozen plasma.

3. The method according to claim 2, wherein the activity of the protein is not less than 80% of the activity of the protein isolated from fresh frozen plasma.

4. The method according to claim 1, wherein IgG isolated from the physiologically active reconstituted spray dried human plasma is isolated in a yield of not less than 80% of the yield in which this protein is isolated from fresh frozen plasma.

5. The method according to claim 2, wherein the activity of the IgG is not less than 80% of the activity of the IgG isolated from fresh frozen plasma.

6. The method according to claim 1, wherein a protein isolated from Fraction IV-1 of the fractionated physiologically active reconstituted spray dried human plasma selected from A1PI, AT-III and a combination thereof is isolated in a yield of not less than 80% of the yield in which this protein is isolated from fresh frozen plasma.

7. The method according to claim 6, wherein IgG isolated front the physiologically active reconstituted spray dried human plasma is isolated in a yield of not less than 80% of the yield in which this protein is isolated from fresh frozen plasma.

8. The method according to claim I wherein albumin isolated from Fraction V of the physiologically active reconstituted spray dried human plasma is isolated in a yield of not less than 80% of the yield in which this protein is isolated from fresh frozen plasma,

9. A composition comprising a member selected from cryopaste and cryo poor plasma prepared by the method according to claim 1.

10. A composition comprising a member selected front Fraction I paste and Fraction I supernatant prepared by the method according to claim 1.

11. A composition comprising a member selected from Fraction II+III paste and Fraction II+III supernatant prepared by the method according to claim 1.

12. A composition comprising a member selected from Fraction IV-I paste and Fraction IV-I supernatant prepared by the method according to claim 1.

13. A composition comprising a member selected from Fraction IV-4 paste and Fraction IV-4 supernatant prepared by the method according to claim 1.

14. A composition comprising a member selected from Fraction V paste and Fraction V supernatant prepared by the method according to claim 1.

15. A preparation of a clotting factor produced by the method according to claim 1.

16. A preparation of IgG produced by the method according to claim 1.

17. A preparation of a member selected from A1PI, AT-III and a combination thereof produced by a method according to claim 1.

18. A preparation of albumin produced by a method according to claim 1.
